(19) United States
(12) Patent Application Publication
(10) Pub. No.: US 2010/0015041 A1
(43) Pub. Date: Jan. 21, 2010

(54) Class of Supramolecular Drug Molecules and Methods of Identification and Use Thereof

(76) Inventor: Gaetano T. Caltagirone, York, PA (US)

Correspondence Address: CLARK & ELBING LLP, 101 FEDERAL STREET, BOSTON, MA, 02110, US

(21) Appl. No.: 11/813,270

(22) PCT Filed: Jan. 17, 2006
(86) PCT No.: PCT/US06/01585

§ 371 Date: Jul. 2, 2007

Related U.S. Application Data
(60) Provisional application No. 60/643,998, filed on Jan. 18, 2005.

(51) Int. Cl. A61K 51/00; A61K 49/00; C07H 21/04; C07H 21/02

(52) U.S. Cl. 424/1.11; 424/9.1; 536/23.1

(57) ABSTRACT

This invention provides a class of supramolecular drugs and methods of identifying and using such drugs

### FIELD OF THE INVENTION

This invention relates to a class of supramolecular drugs and methods of identifying and using such drugs.

### BACKGROUND OF THE INVENTION

The pharmaceutical industry has had success in recent years in generating highly effective, life-saving drugs useful for the treatment of many diseases. Nonetheless, there remains a great need for drugs with increased efficacy and safety to treat these and many additional diseases. For a drug to be effective, it must bind to a target site. However, the majority of drugs exhibit at least some cross-reactivity with other, non-intended sites. In many cases, this results in acceptable side effects, the discomforts of which are outweighed by the therapeutic benefits of the drugs. In other cases, the therapeutic benefits do not outweigh the side effects or risks, leaving patients in need of treatment.

The screening of candidate compounds for safe, effective drugs according to traditional approaches is extraordinarily time-consuming and uncertain. The probability of identifying a hit compound from, for example, a library of random compounds is dependent upon factors such as the size of the library, the nature of the target, and the effectiveness of the screening procedure. Further, even successful in vitro or in vivo assay results are not necessarily predictive of efficacy of a drug candidate in humans. Critical parameters such as absorption, distribution, metabolism, excretion, and toxicity (ADMET) of the candidate are unknown at this stage in the drug development process. As a result, determining the potential effectiveness of a candidate drug requires testing in animal models. Unfortunately, the leap from an in vitro or cell-based assay to demonstration of efficacy in animals almost always requires modification of a compound to improve its therapeutic profile. However, such modifications to original drug chemistry may make a drug ineffective by, for example, reducing its biophysical properties of affinity and specificity and/or by increasing its cross-reactivity and side effects, which, in effect, may destroy the potential of the compound to be an effective drug. Because of all of these uncertainties, traditional drug development is often reduced to a trial and error process, inevitably making it highly inefficient and thus expensive. Indeed, on average, only one in a thousand drug candidates even makes it to human clinical trials. As a consequence, the costs associated with traditional methods of drug development are high ($800+M/drug) and the process is typically quite slow (>6 years/drug).

One of the most common reagents in biological research laboratories, considered an indispensable tool for research, is the oligonucleotide, which is a short, single-stranded nucleic acid molecule (e.g., DNA or RNA). Typically, oligonucleotides are used for antisense research to block gene expression¹, as primers for PCR to amplify minute quantities of DNA², and as components of microarray chips to analyze complex genetic profiles^{3,4}. Familiarity with these oligonucleotide uses has stereotyped these biopolymers as being limited to a certain class of applications, involving Watson-Crick base-pairing. However, oligonucleotides have also been observed to exhibit intriguing functionalities that are completely different from and unrelated to their abilities to hybridize. In particular, libraries of randomized oligonucleotides have been shown to contain oligonucleotide ligands that bind tightly to various target compounds other than nucleic acid molecules having complementary sequences, revealing that oligonucleotides can have binding activities beyond those specified by Watson-Crick base-pairing^{5,6,7,8}.

RNA and single-stranded DNA biopolymer molecules can form a great diversity of structures by exploiting secondary and tertiary interactions, including nonstandard base-pairs, hairpin loops, bulges, multistem junctions, pseudoknots, and four-stranded G-quartet structures. Such biopolymers have structural specificity based on their shapes, and can be engineered to bind to many different compounds, proteins, and molecules. The affinity is imparted by the interplay between structural interactions of the biopolymer with its target, and is similar to the binding of an antibody to a corresponding antigen. For the past fifteen years, a variety of these tight binding nucleic acid ligands, which are generally known as “aptamers,” have been developed and touted as an attractive alternative to conventional antibodies. One reason for the tremendous interest generated by aptamers is the practical advantages of aptamers over antibodies. In cell culture experiments and animal studies, for example, it has been demonstrated that aptamers exhibit neither intrinsic toxicity nor immunogenicity^{9,10}. Further, there is no need to “humanize” an aptamer. Moreover, unlike the time and costs associated with producing protein-based antibodies, aptamers can be synthesized quickly and inexpensively using automated oligonucleotide synthesizers, as most tight-binding aptamers normally range from 25-80 nucleotides in length, well within the capacities of oligonucleotide synthesizers.

Aptamers have been generated without difficulty that bind to organic dyes, drugs, amino acids, nucleotides such as ATP, vitamins, pharmacologically important proteins such as substance P¹¹, the anticoagulant thrombin¹², growth factors, proteases, and several other small and large proteins and enzymes^{5,18,13}. Synthetic combinatorial methods have yielded a DNA ligand that inhibits infection by HIV in vitro²⁰ and a high affinity RNA ligand that inhibits infection by the Human Rhinovirus 14 for the common cold¹⁴. Such methods have also been used to generate antagonists of blood clotting and angiogenesis in tumor growth¹⁸. Some aptamers that have been isolated exhibit stereoselectivity¹⁵. In addition, aptamers have been generated that exhibit greater than 10,000-fold binding affinity for theophylline over caffeine, which differ from one another in structure by only a single methyl group^{19,20}. Another in vitro selection experiment generated an aptamer that binds to D-tryptophan with 670-fold greater affinity than to its counterpart L-tryptophan²⁰. The first aptamer tested in animals was a DNA aptamer that efficiently blocks the proteolytic activity of thrombin. In a canine cardiopulmonary bypass model, this aptamer led to rapid anticoagulation and successfully replaced heparin¹⁶. More recently, an RNA aptamer has been described that reversibly antagonizes the blood clotting activity of coagulation factor IXa¹⁷.

A growing body of literature supports the fact that aptamers exhibit high affinity binding to their cognate targets, and reinforces the notion that an aptamer can potentially be developed against virtually any target. There are a variety of reports of aptamers that exhibit binding affinities from micromolar to nanomolar ranges against various targets¹⁹. In many cases, the binding affinities are observed to be much greater than those of monoclonal antibodies, lead compounds obtained from random peptide libraries, small molecule libraries, and natural product extracts^{18,19}. The selective binding affinity towards targets arises from complex interactive forces characteristic of the nucleotides. Specific interactions such as hydrogen bonding and phosphate group associations dictate the sequence-specific, 3-dimensional structures of the aptamer oligonucleotide ligands, providing a rigid scaffold for the arrangement of aptamer chemical surfaces for target interaction, and lowering the entropic cost of binding as compared to an induced fit interaction^{20,21}.

Even with all the acclaim for aptamers, all nucleic acid biopolymers, including aptamers, are limited by the diversity of the four member genetic alphabet of which they are made: adenine, guanine, thymine/uracil, and cytosine. The binding interactions and chemical reactions of aptamers are constrained by these four functional groups. Thus, even considering the results achieved using traditional drug discovery methods, as well as improvements provided by aptamer technology, there remains a great need for new, effective, and safe drugs to treat a wide variety of medical diseases and conditions, as well as improved methods for identifying such drugs.

### SUMMARY OF THE INVENTION

The invention provides methods for identifying one or more candidate therapeutic or diagnostic molecules. One step of the methods of the invention involves introducing a population of nucleic acid molecule complexes into a test subject (e.g., an animal model of a disease or condition, or a patient, such as a human patient, having or at risk of developing a disease or condition) exhibiting a phenotype of a disease or condition. The nucleic acid molecule complexes can each include (a) a first nucleic acid molecule that includes a segment of random sequences (optionally, flanked by constant segments that facilitate amplification), and (b) a second nucleic acid molecule that includes (i) a nucleic acid molecule segment that hybridizes to the first nucleic acid molecule, and (ii) a functional group conjugated to the nucleic acid molecule segment, which can include one or more nucleotides that establish a fixed spatial orientation of the functional group with respect to the nucleic acid segment. A second step of the methods of the invention involves isolating from the test subject those nucleic acid molecule complexes that localize to a tissue exhibiting a phenotype characteristic of the disease or condition, thereby identifying nucleic acid molecule complexes that are candidate therapeutic or diagnostic molecules for the disease or condition.

In the methods of the invention, the disease phenotype can be characterized by naturally occurring or artificially induced phenotypic markers (e.g., biological, histochemical, immunological, chemiluminescent, physiochemical, radioisotope, photochemical, or electromagnetic markers) that reveal anatomical, physiological, cellular, or receptor structures. Determination of whether a nucleic acid molecule complex has localized to a tissue exhibiting a phenotype characteristic of the disease or condition can involve detection of such a marker by use of imaging reagents.

The methods of the invention can also include a step involving contact of a population of first nucleic acid molecules with a population of second nucleic acid molecules under conditions that promote hybridization, to generate the nucleic acid molecule complexes that are administered to the test subject.

Also, the methods of the invention can include a step of introducing the nucleic acid molecule complexes into a healthy subject, discarding any of the complexes that remain bound or absorbed in the healthy subject, and introducing any remaining complexes into the test subject.

Further, the methods can involve, after isolation of the complexes, (i) incubating the complexes under conditions that disrupt the hybridization between the first and second nucleic acid molecules, and (ii) amplifying the isolated first nucleic acid molecules to generate an amplified, selected pool of first nucleic acid molecules, which can, optionally, be contacted with a population of second nucleic acid molecules to generate nucleic acid molecule complexes for further in vivo selection.

The methods can also, optionally, further include determining the ratio of absorption of nucleic acid molecule complexes in the test subject as compared to a healthy subject. In such an approach, a ratio of greater than one indicates the identification of candidate therapeutic or diagnostic molecules for the disease or condition, which may be subject to further selection.

Further, in another example, the methods of the invention involve subjecting the identified candidate therapeutic or diagnostic molecules to mutagenesis, followed by further in vivo selection, to identify additional candidate therapeutic or diagnostic molecules.

In another example, the isolated nucleic acid molecule complexes are tested for efficacy as therapeutic or diagnostic agents. In another, related example, single, isolated complexes are identified as therapeutic or diagnostic molecules.

The first nucleic acid molecules used in the methods of the invention can include nucleotides (e.g., nucleic acid analogs or derivatives) that are stable from degradation in vivo. At a minimum, the nucleic acid molecules are sufficiently stable, for a sufficient length of time, in vivo for therapeutic action and/or diagnostic detection to take place prior to degradation and/or elimination. As non-limiting examples, such nucleotide analogs can be selected from the group consisting of phosphorothioate, boranophosphate, methyl-phosphonate, and 2′-O-methyl analogs, and analogs thereof. As a preferred example, the analog can be 2′-deoxy-2′-fluoro-RNA (2′-F-RNA).

The second nucleic acid molecules can each include a locked nucleic acid molecule (LNA), a peptide nucleic acid molecule (PNA), a morpholino oligonucleotide, or another type of DNA or RNA molecule. The functional groups of the second nucleic acid molecules can be, for example, independently selected from the group consisting of amino acids, fatty acids, polyethylene glycols, carbohydrates, toxins, small organic molecules, drug compounds, vitamins, cofactors, inorganic compounds, metals, elements of the periodic table and isotope forms thereof, and aggregate or higher-order forms thereof (e.g., peptides, proteins, lipids, complex sugar moieties, or chelated metals). In other examples, the functional groups of the second nucleic acid molecules are independently selected from the group consisting of cytokines, immune response factors, antibodies, complement components, hormones, neurotransmitters, naturally occurring chemicals, and biological macromolecules.

In addition, the nucleic acid molecule complexes can optionally be formed in the presence of one or more metal ions, or one or more organic or inorganic co-factors (e.g., MgCl₂, FeCl₂, CuCl₂, KCl, LiCl, NaCl, RbCl, CaCl₂, SrCl₂, CdCl₂, CoCl₂, MnCl₂, NiCl₂, ZnCl₂, and other elemental salts).

The disease or condition can be, for example, pathological, physiological, psychological, behavioral, or cognitive in origin. As examples, the disease or condition can be selected from the group consisting of cell, tissue, or organ injury, cytopathology, inflammation, repair, regeneration, and fibrosis, immunopathology, neoplasia, developmental disorders, hemodynamic disorders, nutritional pathology, and diseases or conditions affecting the blood vessels, heart, respiratory system, gastrointestinal tract, liver and biliary system, pancreas, kidney, male or female reproductive systems, breast, blood and lymphoid organs, endocrine system, embryonic system, skin, head and neck, bones and joints, skeletal muscle, nervous system and brain, eye and sensory systems such as vision, taste, smell, hearing, and touch.

In other examples, the disease or condition is genetically based, or due to a viral, bacterial, microbial, protozoal, parasitic, or fungal infection. Further, in other examples, the disease or condition is due to diet, poison, toxins, heavy metals, or exposure to environmental hazards.

Specific examples of diseases or conditions include cancer, such as Non-Hodgkin's lymphoma, leukemia, melanoma, lung cancer, pancreatic cancer, stomach cancer, throat cancer, and glioblastoma, Gaucher disease, Hepatitis B and C, respiratory syncytial virus infection, diabetes, obesity, neurological diseases, such as Alzheimer's disease and Parkinson's disease, cardiovascular diseases, such as coronary artery disease, myocardial ischemia, stroke, pulmonary embolism, restenosis, and hypertension, tuberculosis, multiple sclerosis, anaemia, respiratory diseases, arthritis, Crohn's disease, cystic fibrosis, neutropaenia, malaria, general or localized pain, depression, anxiety, psychiatric disorders such as schizophrenia, epilepsy, narcolepsy, sleep disorders, language disorders and aphasias, learning and memory disorders, degenerative disorders, drug addiction, asthma, allergies, gout, hypertension, hypercholesterolemia, dyslipidemia, peptic ulcers, gastroesophageal reflux disease, inflammatory bowel disease, urinary tract infections, and complications of these diseases or conditions.

Nucleic acid molecule complexes identified according to the methods of the invention can be identified as therapeutic or diagnostic molecules that fall into, for example, one or more of the following classes: enzymes, molecular switches, receptor agonists or antagonists, ion channel modulators, anesthetics, analgesics, hypnotics, sedatives, hallucinogenics, cognitive enhancing agents, anti-inflammatory agents, antipyretics, diuretics, antihypertensives, antiarrhythmics, prokinetics, antiemetics, antimicrobials, antifungals, antivirals, antiretrovirals, antineoplastics, immunomodulators, immunosuppresives, immunostimulants, hematopoietic agents, anticoagulants, thrombolytics, antiplatelet agents, antithyroid agents, hormone agonists or antagonists, hormonal-mimetics, dermatological agents, opthalmological agents, toxicological agents, antivenoms, and antidotes.

The invention also includes nucleic acid molecule complexes as described above. These complexes include: (i) a first nucleic acid molecule, and (ii) a second nucleic acid molecule hybridized thereto. The second nucleic acid molecule includes one or more nucleotides that establish a fixed spatial orientation of the functional group with respect to the nucleic acid segment of the second nucleic acid molecule, and further includes a functional group conjugated to the second nucleic acid molecule.

The first nucleic acid molecules can include nucleotides (nucleic acid analogs or derivatives) that are stable from degradation in vivo. As discussed above, at a minimum, the nucleic acid molecules are sufficiently stable, for a sufficient length of time, in vivo for therapeutic action and/or diagnostic detection to take place prior to degradation and/or elimination. For example, the first nucleic acid molecule can include a nucleotide analog selected from the group consisting of phosphorothioate, boranophosphate, methyl-phosphonate, and 2′-O-methyl analogs, and analogs thereof.

In a preferred example, the analog can be 2′-deoxy-2′-fluoro-RNA (2′-F-RNA).

The second nucleic acid molecules can each include a locked nucleic acid molecule (LNA), a peptide nucleic acid molecule (PNA), a morpholino oligonucleotide, or another type of DNA or RNA molecule. Further, the functional groups of the second nucleic acid molecules can be independently selected from the group consisting of amino acids, fatty acids, polyethylene glycols, carbohydrates, toxins, small organic molecules, drug compounds, vitamins, cofactors, inorganic compounds, metals, elements of the periodic table or isotopic forms thereof, and aggregate or higher-order forms thereof (e.g., peptides, proteins, lipids, complex sugar moieties, or chelated metals). In other examples, the functional groups of the second nucleic acid molecules are independently selected from the group consisting of cytokines, immune response factors, antibodies, complement components, hormones, neurotransmitters, naturally occurring chemicals, and biological macromolecules.

Optionally, the nucleic acid molecule complexes are formed in the presence of one or more metal ions, or one or more organic or inorganic co-factors (e.g., MgCl₂, FeCl₂, CuCl₂, KCl, LiCl, NaCl, RbCl, CaCl₂, SrCl₂, CdCl₂, CoCl₂, MnCl₂, NiCl₂, ZnCl₂, and other elemental salts).

The nucleic acid molecule complexes can be identified using, for example, the methods described above an elsewhere herein. Preferably, the nucleic acid molecule complexes have a desired property or activity as described herein, or can be candidates or precursors for molecules having such properties.

The invention also includes methods of preventing, treating, or diagnosing a disease or condition in a patient, by administering to the patient a nucleic acid molecule complex as described herein, which may be identified using the methods described above and elsewhere herein. In one example, the nucleic acid molecule complexes used in such methods were further identified as reducing a marker of the disease in an animal model of the disease. Further, the invention includes methods of enhancing a condition in a patient, such as a condition selected from the group consisting of muscle strength, sexual potency, energy levels, emotional stability, and cognitive or memory capacity, by administering to the patient a nucleic acid molecule complex as described herein, which may be identified using the methods described above and elsewhere herein.

The invention provides several advantages, many of which are due to the fact that molecules identified using the methods of the invention are selected in vivo. In traditional drug discovery, it is only after significant investment made in cell-based and in vitro assays that a candidate drug can be tested in an in vivo system, where the greater complexities of the system may reveal a problem with the candidate, requiring extensive re-design or even complete abandonment. In contrast, according to the present invention, candidate drug compounds are initially screened in an in vivo system, enabling the identification of molecules showing therapeutic or diagnostic promise in the context of a complex living system from the outset. Thus, the methods of the invention facilitate the early identification of agents that have a much greater likelihood of being safe and effective in organisms such as humans than other approaches, resulting in greater efficiency. Such increased efficiency can provide enormous medical and societal benefits, including decreased time required for effective, safe drug development, as well as decreased costs.

Additional advantages of the invention relate to the fact that drug agents identified according to the invention are based on oligonucleotide molecules, which are easy and inexpensive to make, particularly as compared to other types of drugs, which may comprise highly complex organic molecules, requiring costly and technically demanding syntheses. Further, as is noted above, nucleic acid-based agents have generally been found to be safe, and safety can be readily assessed in the initial, in vivo screen. In addition, the molecules of the invention are relatively small compared to therapeutic antibodies, thus facilitating their circulation through the body and penetration of tumors and other disease targets.

Other advantages of the invention relate to the fact that, as discussed below, the molecules can be selected based on their association with/localization to a tissue associated with a specific disease phenotype. Such selection requires only knowledge of a phenotype associated with a target disease or condition, and not information as to potential disease causes at a molecular level, as is required in some more traditional drug development schemes. The invention can thus be summarized as providing rapid, effective, and efficient approaches to identifying specific, safe compounds for use in therapeutic and diagnostic methods.

Other features and advantages of the invention will be apparent from the following detailed description, the drawings, and the claims.

### BRIEF DESCRIPTION OF THE DRAWINGS

FIG. 1 is a schematic representation of aptabody molecular structures. Aptabody molecules are supramolecular structures consisting of one or more functional groups, which may be spatially oriented, attached to an oligonucleotide scaffold. Pharmacological activity can arise from the precise positioning of these functional groups within 3-dimensional space. Characteristics such as absorption, distribution, metabolism, excretion, and toxicity (ADMET) are predetermined by the unique combination and interplay of these various functional groups.

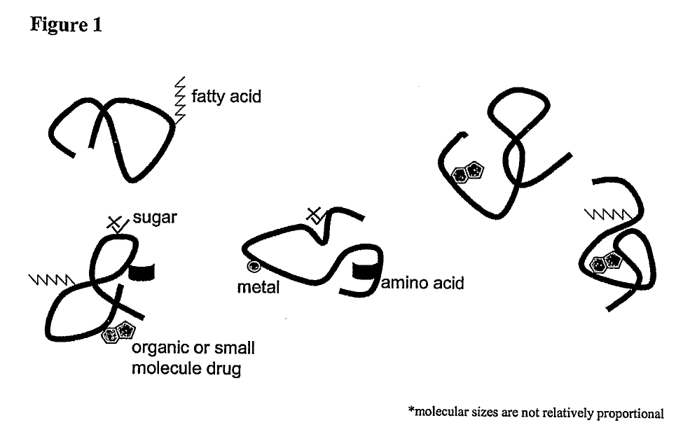

FIG. 2 is a schematic representation of the assembly of an aptabody Library. Freshly prepared radiolabeled 2′-F-RNA is mixed with tLNAs in a 1:1 ratio, and PBS buffer (pH 7.4) containing 1 mM each of MgCl₂, FeCl₂, CuCl₂, KCl, LiCl, NaCl, RbCl, CaCl₂, SrCl₂, CdCl₂, CoCl₂, MnCl₂, NiCl₂, and ZnCl₂. The sample mixture is incubated at 37° C. overnight in a closed microcentrifuge tube to allow for hybridization and molecular folding into thermodynamically stable aptabody molecules. Locked nucleic acid oligomers readily strand invade duplex regions and exhibit melting temperatures greater than 60° C.

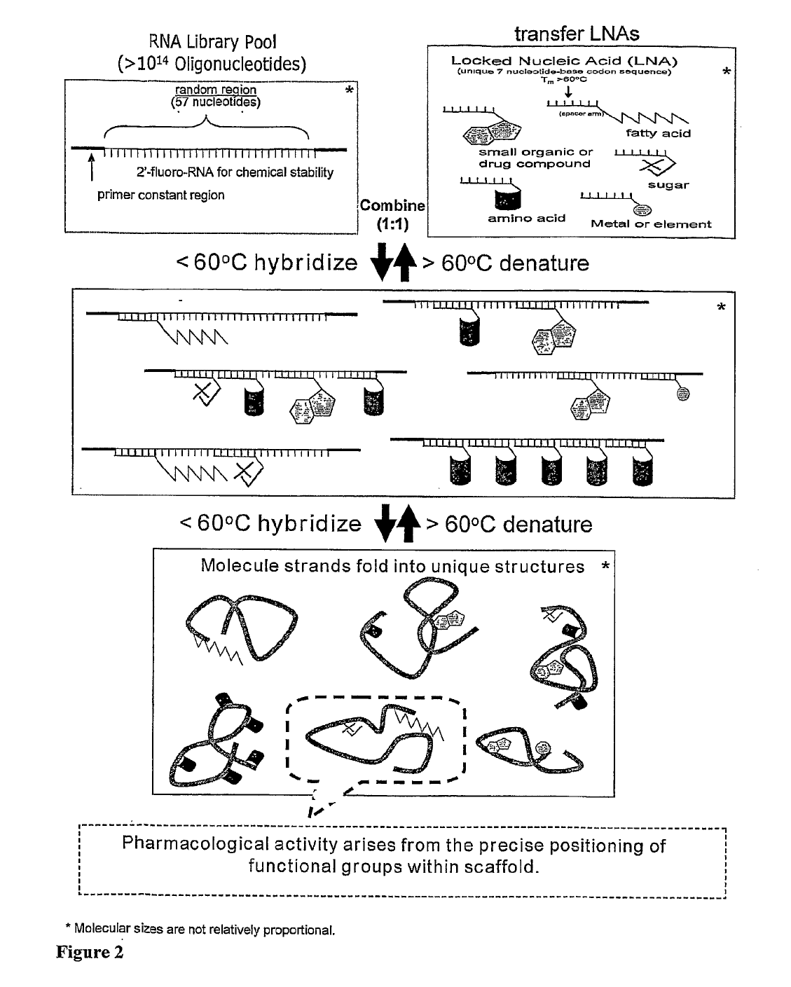

FIG. 3 is a schematic representation of exemplary transfer LNA (tLNA) functional groups conjugated to unique LNA sequences. Functional groups containing carbonyl groups react with the 5′-end of an amine-modified 7-mer LNA in the presence of hydrazine. Functional groups containing primary amine groups react in the presence of SANH-amine reactive hydrazine and SFB-amine reactive aldehyde reagent. The unique LNA sequences are designed to be non-interfering with each other.

FIG. 4 is a schematic representation of the selection of highly specific aptabodies in a metastatic tumor model. A library of aptabodies is pre-exposed in a negative selection step to a normal, healthy mouse, followed by positive selection in a mouse tumor model to maximize specificity to tumor tissue. As selection progresses, the library pool is enriched with aptabodies specific to tumors. These aptabodies are then screened for pharmacological activity.

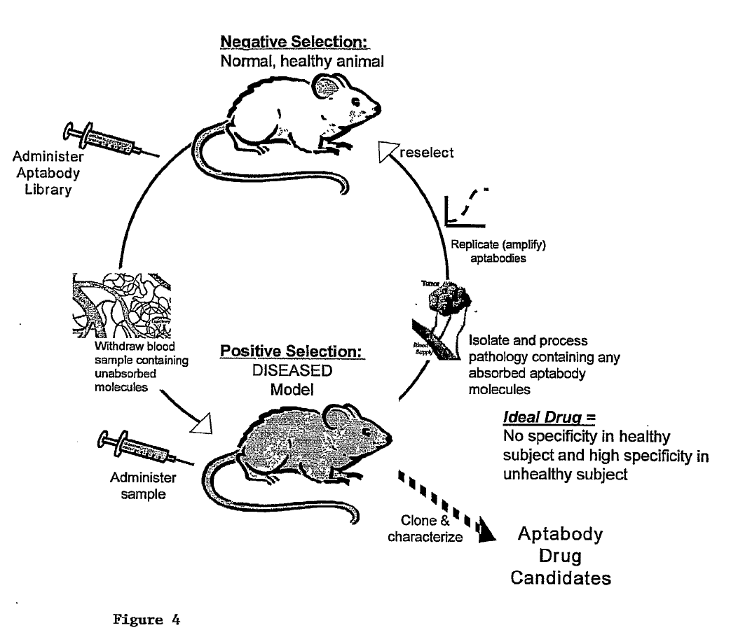

FIG. 5 is a schematic representation of tissue sectioning and isolation of aptabodies associated with the histopathological marker in a single tissue sample (A) and in several generations of in vivo selection (B).

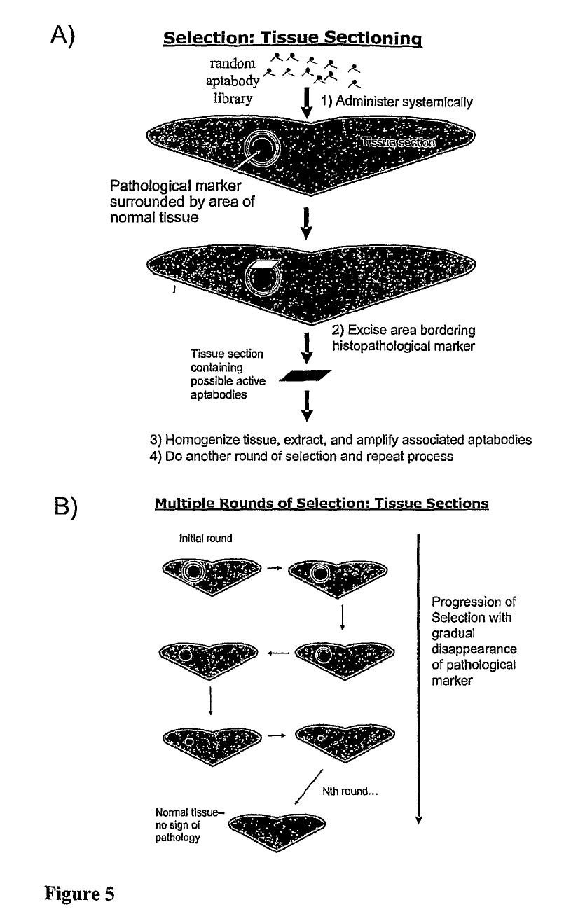

FIG. 6 is a schematic representation of the molecular switch technology of the invention, in which the functional group component of a nucleic acid molecule complex of the invention is an anticancer drug, the activity of which is blocked systemically and activated at a specific target area, due to exposure of the active site.

FIG. 7 is a schematic representation of the molecular switch technology of the invention, in which the functional group component of a nucleic acid molecule complex of the invention is a thrombolytic enzyme, the activity of which is blocked systemically and activated under ischemic conditions, due to exposure of the active site.

FIG. 8 is a schematic representation of the molecular switch technology of the invention, which shows a gradient of an unknown effector molecule emanating from the site of an occlusion, which converts an inactive drug into an active drug by exposure of the active site.

### DETAILED DESCRIPTION

The present invention provides a new class of pharmaceutical molecules, as well as methods of making, screening, and using these molecules as therapeutic and/or diagnostic agents. The molecules of the invention, which are referred to herein as “aptabodies,” are supramolecular-modified aptamers. These nucleic acid molecule complexes thus include a nucleic acid, aptamer component, to which is bound one or more functional groups (e.g., fatty acids, polyethylene glycols, amino acids, carbohydrates, and/or other organic molecules; see below). As is discussed further below, the attached functional groups provide an enormous source of structural complexity and chemical reactivity, thus leading to functional diversity of the molecules.

Central to the invention is the method by which the aptabody molecules are identified. In particular, the screening methods of the invention, which are discussed in further detail below, involve iterative cycles of in vivo selection of molecules having particular localizations and/or activities from aptabody libraries, in which the aptamer portions of the aptabodies include random sequences. The functional diversity of the molecules of the invention, as well as the fact that they are selected from diverse libraries in a physiologically relevant environment, highlights the power of this system. The molecules and methods of the invention are described in further detail below, followed by an example of application of the methods of the invention in a mouse tumor model.

### Aptabodies

### The Nucleic Acid Component

As is noted above, aptabodies comprise a nucleic acid component, similar to an aptamer, as well as one or more functional groups attached to the nucleic acid portion of the molecule. It is the nucleic acid, aptamer component that enables the amplification of selected aptabody molecules as described herein because, as is well known in the art, such molecules can be amplified using processes such as the polymerase chain reaction (PCR).

The nucleic acid, aptamer molecule component of aptabody molecules can comprise nucleotides of ribonucleic acid (RNA), deoxyribonucleic acid (DNA), or any modified or stabilized forms of such nucleotides (or combinations thereof), provided that the nucleotides can be incorporated into polymers by polymerase enzyme systems and, when in the form of polymers, be read as templates for further synthesis by these systems. Preferably, the nucleotides are modified so as to reduce or eliminate the possibility of their degradation in vivo. As examples, phosphorothioate, boranophosphate, methyl-phosphonate, or 2′-modified analogs can be used. A specific example of the latter type of analog that can be used in the invention, and which is described further below, is 2′-deoxy-2′-fluoro-RNA (2′-RNA). Other examples of such analogs that can be used are 2′-O-methyl-RNA and 2′-amino-RNA. Synthesis of molecules including these and other nucleotides that result in increased stability is known to those of skill in the art. The required nucleotides, as well as oligonucleotides that include them, are available from a variety of commercial sources (e.g., TriLink Biotechnologies, San Diego, Calif., U.S.A.; Proligo LLC, Boulder, Colo., U.S.A.; Glen Research, Sterling, Va., U.S.A.; Oligos etc., Wilsonville, Oreg., U.S.A.).

As is noted above, the nucleic acid molecule portion of the aptabodies in the libraries that are screened according to the methods of the invention are designed to include a stretch of random sequences (e.g., 10-100, 20-80, 30-70, or 40-60 nucleotides in length). To facilitate the selection and amplification process, the stretch of random sequences is flanked with constant sequences that are used as primer binding sites for reverse transcription (in the case of RNA aptabodies) and amplification by the polymerase chain reaction (PCR). The primer sequences can consist of 7-40, e.g., 10-30 or 15-25, nucleotides that are complementary to the constant sequences noted above. One of the primers also advantageously includes a promoter sequence for an RNA polymerase, such as T7 RNA polymerase, which facilitates reverse transcription of the library and/or selected molecules into RNA molecules for further selection and/or for use as therapeutic or diagnostic agents. Such oligonucleotides, including sections of random sequence, can be made using methods that are known in the art, and also can be obtained from a variety of commercial sources (see, e.g., above).

### The Functional Group Component

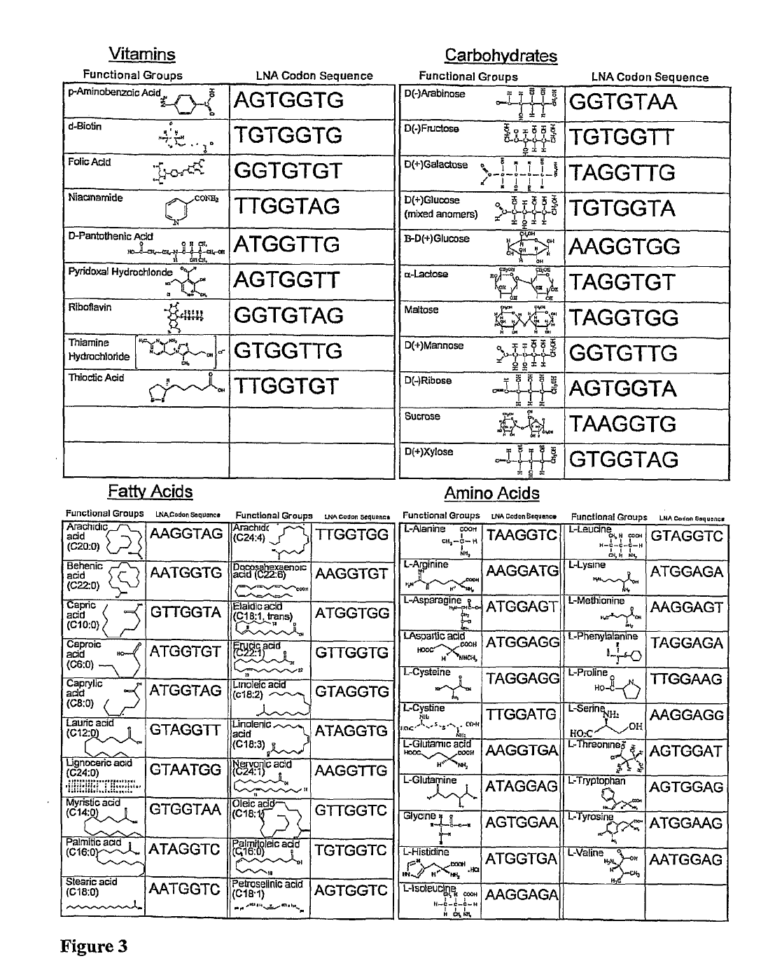

In addition to the nucleic acid component described above, aptabody molecules of the invention include one or more non-nucleic acid molecule functional groups. These functional groups can include virtually any type of molecule including, for example, any type of amino acid, fatty acid, carbohydrate, toxin, small organic molecule, drug compound, vitamin, cofactor, inorganic compound, polymer such as polyethylene glycol, synthetic or natural compound, metal, element of the periodic table or isotopic form thereof, or any modified versions of such molecules and/or combinations thereof. In other examples, aggregates or polymers of such molecules can be used including, e.g., peptides, proteins, lipids, complex sugar moieties, and chelated metals. In other examples, the functional groups of the second nucleic acid molecules are independently selected from the group consisting of cytokines, immune response factors, antibodies, complement components, hormones, neurotransmitters, naturally occurring chemicals, and biological macromolecules. Specific examples of functional groups that can be used in the invention include those shown in FIG. 3, which lists examples of vitamins, carbohydrates, fatty acids, and amino acids that can be used. Additional examples of functional groups are provided in Example II, below. The only requirement of a functional group used in the construction of an aptabody library according to the invention is that it can be linked to the nucleic acid molecule portion of the aptabody molecule, as described herein.

The functional groups can impart some type of activity on the aptabodies, such as an enzymatic activity. In other examples, the functional groups play a role in determining the bioavailability and/or targeting of the aptabodies in vivo. As is discussed further below, selection can be carried out until a desired activity arises. Nonetheless, in the case of aptabodies found to localize to a particular target tissue, but to lack any type of activity, further modification of the molecules can be carried out to impart a desired activity. For example, in the case of an aptabody that targets a cancerous tissue, the aptabody can be modified to include a component that will result in destruction of the tissue to which it is targeted. In other examples, an aptabody found to target a particular tissue can be used to deliver agents that can be used in imaging methods. Further, an aptabody found to localize to a target tissue, but to otherwise be inert, can be subject to mutagenesis, in an effort to identify molecules that maintain specific localization, but which gain a desired activity.

Because of the need to amplify the nucleic acid molecule portion of the aptabody molecules during selection, it is essential that the functional group component does not interfere with amplification. This can be achieved by having the functional group linked to the nucleic acid component by a linker or transfer molecule having the following properties: (i) it can be removed from the nucleic acid molecule component to enable amplification, (ii) it can direct specific reattachment of the functional group after amplification, and (iii) it can ensure that the functional group component remains attached to the nucleic acid component of the aptabodies in vivo. Linkers/transfer molecules having these properties include nucleic acid molecule-based linkers and, in particular, nucleic acid-based linkers that can hybridize to the nucleic acid molecule component of the aptabodies with enhanced stability.

In some examples, and as is discussed further below, the functional groups in the molecules of the invention can be positioned such that their spatial orientation relative to the nucleic acid portion of the molecule is fixed. In other examples, the positions of the functional groups are not rigidly set, thus allowing a wide range of activities in a single molecule. In any case, the positioning of the functional groups within three-dimensional space can give rise to pharmacological activity. Further, properties of the molecules such as absorption, distribution, metabolism, excretion, and toxicity (ADMET) can be set by the unique combination and interplay of the various functional groups.

One example of a linker or transfer molecule that can be used in the invention is one based on Locked Nucleic Acid™ (LNA®) molecules, which are nucleic acid analogs that contain a 2′-O, 4′-C methylene bridge. This bridge restricts the flexibility of the ribofuranose ring and locks the structure into a rigid C3-endo conformation, resulting in enhanced hybridization and biological stability. LNA molecules can be obtained from a variety of commercial sources (Exiqon, Vedbaek, Denmark; Proligo LLC, Boulder, Colo., U.S.A.; Synthegen, Houston, Tex., U.S.A.).

Another example of a linker/transfer molecule that can be used in the invention is based on Peptide Nucleic Acid (PNA) molecules, which are analogs of DNA in which the backbone is a pseudopeptide rather than a sugar. PNA mimics the behavior of DNA and binds to complementary nucleic acid strands. The neutral backbone of PNA results in stronger binding and greater specificity than normally achieved. PNAs can be synthesized using methods known in the art and are also available from commercial sources (Active Motif, Carlsbad, Calif., U.S.A.)²².

Other types of nucleic acid molecule linkers/transfer molecules can also be used, with a desirable feature being high melting temperature (e.g., greater than 37° C., preferably greater than 55, 60, or 65° C.), to assure stability in vivo. Thus, in addition to LNAs and PNAs, oligonucleotides such as morpholino oligonucleotides, which comprise nucleotides that are linked to a six member morpholine backbone (Gene Tools LLC, Philomath, Oreg., U.S.A.), can be used in the invention. The LNA and PNA linkers can include sequences ranging from, for example, 5-15, 7-12, or 9-11 nucleotides for binding to the aptamer portion of the aptabody molecules. As is known in the art, selection of an appropriate sequence length for binding will depend upon, for example, the nature of the nucleotides (e.g., whether LNAs or PNAs are used), the length of the nucleic acid molecule component, the desired level of variation in the library, as well as the nucleotide content of the hybridizing region (i.e., G/C vs. A/T content).

Connection of functional groups to the linker molecules can be carried out using standard methods, which will vary depending upon the natures of the functional groups and the linkers. As two examples, functional groups containing carbonyl groups react with the 5′-end of an amine-modified LNA in the presence of hydrazine, and functional groups containing primary amine groups react in the presence of SANH-amine reactive hydrazine and SFB-amine reactive aldehyde reagent. Other approaches for making these and other types of linkages are known in the art. In addition, kits for preparing such functional group/linker molecules, conjugation services, as well as such conjugated molecules themselves, are available from commercial sources (e.g., Solulink, Incorporated, San Diego, Calif., U.S.A.; Pierce Biotechnology, Inc., Rockford, Ill., U.S.A).

### Co-Factors

Additional functionality and diversity of aptabody molecules of the invention can be obtained by the use of one or more metal ions, organic co-factors, and/or inorganic co-factors. As examples, biologically significant metal ions such as MgCl₂, FeCl₂, CuCl₂, KCl, LiCl, NaCl, RbCl, CaCl₂, SrCl₂, CdCl₂, CoCl₂, MnCl₂, NiCl₂, and ZnCl₂, alone or in combination, can be used. Many of these metal ions are important constituents of natural enzyme systems, and may be important for any number of these supramolecular structures formed. Functional aptabodies may require one or more metal ion cofactors for structural stability and activity, or for maximizing activity. The metals noted above (or others) can be incorporated into the aptabodies by, for example, including them in the reaction mixture when aptabodies are assembled or contacting the aptabodies with them after formation.

Methods for identifying and using aptabodies are described below. The invention includes aptabodies identified using these or other methods.

### Selection Methods

The methods of the invention begin with the construction of a library of aptabody molecules, as described above, which is subject to selection as described herein, until one or more molecules having desired characteristics is isolated. As is noted above, the aptabodies in the initial library, which may include, e.g., 10¹² to 10¹⁵ different molecules, have a nucleic acid molecule component that includes a segment of random sequences, as well as fixed sequences that can be used as primer binding sites for amplification of selected molecules. The aptabodies also include one or more functional group components as described above, which are linked to the nucleic acid component in such a way that facilitates removal for amplification, yet provides stability in a physiological context.

An initial library of aptabody molecules is prepared by transcription of a pool of oligonucleotides including a segment of random sequences flanked by constant sequences, as described herein, incorporating nucleotides that provide enhanced stability to the transcribed molecules. Functional group components are then added to the nucleic acid molecule components using approaches appropriate for the given functional groups, as determined by those of skill in the art, optionally, in the presence of any desired co-factors. The library of assembled aptabody molecules is then administered to a test subject (e.g., an animal model of disease or condition; see below) and those molecules localizing to a target tissue (in the case of positive selection; see below) are extracted from a homogenate of the tissue. Functional group components are then removed by incubating the selected molecules at or above the melting temperature of the hybrid formed between the nucleic acid component of the aptabody and the linker to the functional group, selected sequences are reverse transcribed into cDNA and amplified by PCR, amplified sequences are transcribed into 2′-F-RNA, and aptabody molecules are reformed by admixture with functional group components as described above, for further selection.

Central to the selection methods of the invention is the use of an in vivo system. As is discussed above, use of such a system enables the isolation of molecules having physiologically relevant properties in the initial stages of drug discovery, in contrast to traditional drug discovery methods, involving extensive in vitro testing that often results in the identification of candidates that prove to be ineffective or otherwise unacceptable in vivo. In general, animal models of disease are used in the invention, but the invention also includes the use of human subjects for the isolation of aptabodies.

Another central feature of the selection methods of the invention is the use of both negative and positive selection. The methods of the invention require that there be a phenotype or tissue localization associated with a disease or condition for which it is desired to identify a therapeutic or diagnostic molecule. Aptabody molecules are selected in the methods of the invention due to their localization to a target tissue exhibiting such a phenotype. To eliminate from the selection process those molecules that localize to the target tissue in subjects not affected by the disease, a negative selection step can be carried out. In such a selection, candidate molecules are administered to a subject not having the disease, and molecules that do not localize to the target tissue (e.g., molecules that remain in circulation) are selected for further amplification and selection in a subject affected by the disease. When administered to subjects exhibiting the disease phenotype, candidate molecules are isolated and amplified from a tissue exhibiting the disease phenotype, for further rounds of selection. In this manner, any candidate molecules that localize to the target tissue in an affected subject are likely to be specific for the tissue in diseased state and not to localize to a corresponding healthy tissue.

Inclusion of appropriate negative and positive selection steps can be determined by those of skill in the art based on factors such as, for example, the relevant disease or condition, the availability of corresponding affected and healthy subjects, and the apparent likelihood of identifying a molecule with desired features. In one example, the first selection can be a negative selection step carried out in a healthy subject, and all subsequent steps can be positive selections, carried out in subjects affected by the disease or condition. In another example, additional negative selection steps can be included. As is stated above, in the negative selection steps, molecules that do not localize to a target tissue are isolated for further amplification and selection, while in positive selection steps molecules that localize to a target tissue are isolated and subject to further amplification and selection.

As is discussed further below, selection can be carried out for several (e.g., 7-15) iterations, after which candidate molecules having desirable localizations/activities can be isolated and characterized. Whether a potentially effective candidate has been identified can be determined by, for example, analysis of the phenotype relevant to the disease or condition at issue. In another variation of the methods of the invention, a pool of candidate molecules found to localize to a target tissue of interest, preferably specifically for the disease model, are subjected to mutagenesis during or subsequent to the entire selection scheme, to create greater diversity, with the potential to generate more molecules having a desired or enhanced activity/localization. Another tool that can be used in the selection process is determination of the ratio of aptabody binding to the target tissue in the disease model as compared to a corresponding healthy animal. Determination of a ratio of greater than one indicates the identity of molecules having specificity for a given target, and the higher the ratio, the greater the binding/specificity.

The methods of the invention can be used to identify aptabody molecules for use in the treatment or diagnosis of any disease or condition having a detectable phenotype in a particular tissue and/or known to be associated with a particular tissue. One example, noted above, is cancer. In particular, aptabody molecules can be identified that specifically localize to a tissue including a tumor and/or to the tumor itself. Numerous animal models of cancer are known in the art, including those in which the cancer is due to transgenic alteration of the genome of the animal, induction by chemical or other means, or implantation of cancerous cells. Specific examples of such animal models include, for example, mice that express high levels of ornithine decarboxylase (ODC), which are highly susceptible to tumor development upon exposure to low doses of carcinogenic compounds, and mice that express the ras sarcoma virus oncogene, which spontaneously develop angiosarcomas and lung adenosarcomas (Taconic, Hudson, N.Y., U.S.A.).

Aptabodies that can be used to treat neurodegenerative diseases can also be identified, according to the invention, using appropriate animal model systems. There are, for example, numerous animal models of Alzheimer's disease, many of which are available commercially. For example, transgenic mice expressing human β-amyloid precursor protein²³ and/or Tau²⁴ can be obtained from Taconic (Hudson, N.Y., U.S.A.). In the case of, for example, Parkinson's Disease, there are several animal models that can be used, including transgenic mouse models^{25,26,27,28} and models in which the condition is chemically induced^{29,30,31,32,33,34}.

In other examples, animal models of cardiovascular disease, induced transgenically, by physical means, and/or by diet, can be used to identify aptabodies that can be used in the treatment of these diseases. For example, mice that have alterations in apolipoprotein, cholesterol ester transferase, and/or peroxisome proliferator activator-α genes exhibit elevated levels of serum low density lipoprotein (LDL), decreased levels of high density lipoprotein, altered fatty acid metabolism, and/or hyperlipidemia, each of which can lead to increased incidence of atherosclerotic lesions. These mice can be used to identify aptabodies that can be used in the treatment of diseases characterized by one or more of these physiological conditions.

In other examples, animal models of coronary artery disease or restenosis after angioplasty treatment, generated by appropriate injury to the model, can be used. As a specific example, the left common carotid artery of a rat can be injured with a 2 F embolectomy catheter (Baxter Healthcare, Deerfield, Ill., U.S.A.), as described³⁵. An aptabody library can then be administered to the rat by, e.g., bolus tail vein injection of 1 ml of a 1.0 mg/ml library preparation diluted in PBS (pH 7.4). Five minutes after library administration, 1.0 ml of 0.5% Evans blue dye (Sigma-Aldrich Corp., St. Louis, Mo., U.S.A.) is administered to the animal by intravenous infusion to allow identification of the occluded vessel segment. The animal is then euthanized, the occluded segment is isolated, any aptabody molecules that have localized to the segment are extracted and isolated, the functional group/linkers are removed from the aptabodies by heating, the nucleic acid molecule components of the aptabodies are reverse transcribed and amplified by PCR, amplified DNA is transcribed into RNA including nucleotides that prevent degradation in vivo (e.g., 2′-fluoro-nucleotides; see above), aptabody molecules are reassembled by incubation of the RNA molecules with functional group/linker molecules, and another round of selection is carried out.

Any other animal model of disease can be used in the screening methods of the invention. Further, even if an animal model is not available for a particular disease, selection of molecules that localize to a tissue known to play a role in a disease can be used to identify candidate therapeutic or diagnostic molecules, which can then be tested directly in patients.

### Therapeutic and Diagnostic Applications of Aptabodies

Over the course of several selection steps, aptabody molecules are isolated that localize to particular target tissues. As is noted above, it is preferable to identify aptabodies that target diseased tissue preferentially over corresponding healthy tissue. Indeed, over the course of selection it is preferable to identify molecules that are both specific for the diseased tissue and which show some type of therapeutic benefit in the tissue. As an example, it is desirable to identify a molecule that both localizes to a tissue that may include a tumor, as well as reduces the size of and/or eliminates the presence of the tumor from the tissue. In other examples, molecules are selected that, rather than targeting a tissue including a tumor, target the tumor itself. In the latter example, it is preferable that an aptabody having a therapeutic effect (i.e., destruction of the tumor) be identified. Nonetheless, if the molecule only shows correct localization but not a therapeutic effect, it can be modified to include an agent that will induce the desired effect, such as a cytotoxic molecule. Further, such molecules, in targeting a tumor and not a particular tissue in which the tumor may be present, can be used to treat metastases of the tumor, localized in other tissues of the body. Molecules identified as localizing to a specific tissue, but not differentiating between diseased and healthy target tissues, can be used to target compounds to these tissues for use in, for example, imaging methods. Further, as is described below in Example II, molecules can be identified according to the invention that are active in specific, target regions of the body (as dictated by, e.g., certain physiological conditions), and inactive elsewhere. In one example of such molecules, the functional group of the molecule is a known drug, which has been modified to be a component of a nucleic acid molecule complex as described herein. In this context, the nucleic acid component of the aptabody molecule acts as a molecular switch, which can be triggered by physiological signals at or near a target site of the drug.

In the case of molecules identified as having therapeutic activity, such molecules can be administered to subjects in need of treatment consistent with the therapeutic activity according to standard methods, which will vary depending on, for example, the location where an effect is required, as well as the activity of the compound and the disease or condition. For example, administration can be systemic or local. Specific, non-limiting examples of approaches that can be used include parenteral routes, such as intravenous, intraarterial, subcutaneous, intradermal, intramuscular, intrathecal, pulmonary, topical, intraventricular, intracapsular, intraspinal, intracisternal, and intraperitoneal routes, as well as mucosal routes, such as oral (e.g., sublingual), ocular, intranasal, rectal, and vaginal routes. Further, the therapeutic molecules of the invention can be administered by the use of catheter systems, which isolate a target tissue. Such an approach can be used, for example, in treating cancer that is isolated to the liver.

A therapeutic molecule of the invention can be administered within a pharmaceutically-acceptable diluent, carrier, or excipient, in unit dosage form. Administration can begin before or after the patient is symptomatic. Methods for making formulations are well known in the art³⁶. Further, determination of appropriate dosage amounts and regimens can readily be determined by those of skill in the art.

The molecules can also be used in diagnostic methods. For example, a molecule can be selected for specifically binding to a target tissue showing characteristics of a disease or condition. Such a molecule can be used in diagnostics if, for example, it is complexed with a label that can be detected using imaging techniques. In addition to being used for delivering detectable labels in this manner, the molecules can also be used to deliver additional therapeutic molecules, such as toxins for use in the destruction of cancer cells.

The invention is described in further detail in the following, non-limiting example.

### EXAMPLES

### Example I

### Aptabody Library Construction and Selection

### Summary

A large combinatorial library is designed consisting of the following aptabody components: chemically diverse functional groups (e.g., amino acids, carbohydrates, fatty acids, polyethylene glycols, and small organics) coupled to random oligonucleotide sequences of the highly stable 2′-deoxy-2′-fluoro-RNA (2′-F-RNA). Selection of aptabody molecules that specifically target tumor tissue is accomplished through negative selection for aptabodies that are unabsorbed in a normal/healthy animal, followed by positive selection for aptabodies that specifically target a pathological marker in an animal model of disease.

A healthy mouse is first challenged with a library size of 10¹⁴ molecules. Unbound or unabsorbed aptabody molecules become the first round of candidate therapeutic molecules. These molecules are then administered to a mouse tumor model. Sacrifice of the animal and extraction of the tumor tissue permits recovery of aptabody molecules that concentrate at the disease site, which are isolated and amplified. Further rounds of challenge, recovery, and amplification reduce the number of candidates to those with the highest affinity to and specificity for tumor tissue. In further selection, pharmacologically active aptabody molecules are selected from those that specifically target metastatic tumors.

### Experimental Design and Methods

### Combinatorial Aptabody Library Construction

Aptabody molecules are created from two physiologically stable components. The first component is a random library of 2′-deoxy-2′-fluoro-RNA (2′-F-RNA), a nuclease resistant and physiologically stable form of RNA³⁷. Unlike proteins and antibodies, or DNA and RNA aptamers, 2′-F-RNA aptamer ligands are resistant to ribonuclease, deoxyribonuclease, and protease degradation. As described in detail further below, in this example, the 2′-F-RNA library is 100 nucleotides in length, designed with a 57-nucleotide stretch of random sequence flanked by 5′ and 3′ constant regions.

The second component, referred to herein as tLNAs (transfer LNAs), is any number of Locked Nucleic Acid (LNA) oligonucleotide sequences, 7 nucleotides long, each coupled to a functional group. Locked nucleic acids are nucleic acid analogs containing a methylene bridge that connects the 2′-oxygen of ribose with the 4′-carbon. This bridge results in a locked 3′ endo conformation that reduces the conformational flexibility of the ribose, allowing for highly stable hybridization to a complementary sequence of DNA or RNA. In fact, because of their rigid conformation, LNAs have dramatically enhanced affinity towards complementary DNA or RNA as compared to DNA and RNA duplexes resulting in an increased melting temperature (Tₘ) of 3° to 8° C. per LNA base (Proligo LLC, Boulder, Colo., U.S.A.). Moreover, LNAs are incredibly stable under physiological conditions and are known to be stable in urine and sera for as many as two days. They have also been shown to improve the in vivo stability of aptamers, without compromising their binding properties³⁸.

The tLNAs and 2′-F-RNA library are combined (or hybridized) through Watson-Crick base pairing to form highly stable duplex structures with melting temperatures of at least −50° C. (e.g., 50° C., 55° C., or 60° C.), but preferably no more than 65° C. This high melting temperature allows sufficient stabilization of the complexes within the 37° C. bloodstream. The tLNA constructs, with their unique seven nucleotide sequences, have a (1/4)⁷ chance of hybridizing to the random region of 2′-F-RNA during aptabody assembly.

As an added component to the hybridization mix, various biologically significant metal ions can be added including, for example, any one or more of MgCl₂, FeCl₂, CuCl₂, KCl, LiCl, NaCl, RbCl, CaCl₂, SrCl₂, CdCl₂, CoCl₂, MnCl₂, NiCl₂, and ZnCl₂. Many of these metal ions are important constituents of natural enzyme systems, and so it is reasonable to predict that any number of the supramolecular structures formed between 2′-F-RNA and tLNA may require one or more metal ion cofactors for structural stability and activity, or have these properties enhanced by such cofactors.

tLNA Synthesis

A 5′-amine labeled LNA (Proligo LLC, Boulder, Colo., U.S.A.) is covalently coupled to the reactive carbonyl group of each compound (Sigma-Aldrich Corp., St. Louis, Mo., U.S.A.) using a hydrazine/carbonyl conjugation system (Solulink, Incorporated, San Diego, Calif., U.S.A.) according to supplier instructions; see FIG. 3. The modified LNA is purified against unreacted LNA by size fractionation. An approximate concentration of each modified LNA, or transfer LNA (tLNA), can be determined by spectrophotometrically at 260 nm. 100 μM stock solutions in PBS buffer (pH 7.4) of tLNA can be stored at −20° C.

### Synthesizing 2′-F-RNA Library

Oligonucleotides used for 2′-F-RNA synthesis can be prepared by automated solid support phosphoramidite chemistry provided by a supplier (Proligo LLC, Boulder, Colo., U.S.A.) and purified by denaturing (8 M urea) PAGE before use to ensure full-length starting material. The DNA template (200 picomoles=10¹⁴ molecules) for pool construction, 5′-GGGCAACCTACGGCTTTCACCGTTTCG-[N₅₇]-CTCATCAGGGTCGCCC-3′ (APTA57) can be made double stranded by extension in the presence of 320 picomoles of APTA-T7 primer, 5′-TAATACGACTCACTATAGGGCGACCCTGATGAG-3′, which contains the promoter for T7 RNA polymer (T7 RNAP). The DNA extension reaction can be carried out using Exo-minus Klenow DNA polymers (Epicentre, Madison, Wis., U.S.A.) according to the manufacturer's instructions. The resulting double stranded DNAs can be recovered by precipitation with ethanol, resuspended in 20 μl of sterile molecular grade water, and stored at −20° C. until needed.

One-fourth of the sample is made into 2′-F-RNA in the DuraScribe® in vitro transcription kit (Epicentre, Madison, Wis., U.S.A.). RNA transcription in the presence of 2′-fluoro-deoxyribonucleotides (TriLink Biotechnologies, San Diego, Calif., U.S.A.) yields nuclease-resistant and stable RNA products³⁹. This is accomplished by completely replacing CTP and UTP with 2′-Fluorine-dCTP (2′-F-dCTP) and 2′-Fluorine-dUTP (2′-F-dUTP) in the in vitro transcription reaction. The presence of the fluorine at the 2′-position of the 2′-F-dC and 2′-F-dU nucleotides prevents RNase A digestion. The Durascribe™ T7 RNA polymerase efficiently incorporates canonical NTPs as well as the non-canonical 2′-F-dCTP and 2′-F-dUTP into RNA. The T7 transcription buffer, together with other reaction components and 5 mM each of the nucleotide mix: 2′-F-dCTP, 2′-F-dUTP, ATP, GTP, is incubated at 37° C. for 6 hours to yield a pool of transcribed RNA. Following DNAse I (Epicentre, Madison, Wis., U.S.A.) treatment at 37° C. for 15 minutes, the RNA is isolated by denaturing 10% PAGE. The RNA band is excised from the gel using UV shadowing as a guide, eluted overnight at 23° C. in 425 μl 1300 mM NaCl, ethanol precipitated, and resuspended in 100 μl PBS (pH 7.4).

### Assembly of Aptabody Library

The freshly prepared, 2′-F-RNA is mixed with tLNAs at a 1:1 ratio, and PBS buffer (pH 7.4) containing 1 mM each of MgCl₂, FeCl₂, CuCl₂, KCl, LiCl, NaCl, RbCl, CaCl₂, SrCl₂, CdCl₂, CoCl₂, MnCl₂, NiCl₂, and ZnCl₂. The sample mixture is incubated at 37° C. overnight in a closed microcentrifuge tube to allow for hybridization and molecular folding of thermodynamically stable aptabody molecules. Locked nucleic acid oligomers readily strand invade duplex regions.

To provide empirical evidence for the existence of hybridization events occurring between 2′-F-RNA and tLNAs, a non-denaturing PAGE analysis is performed on known sequences of 2′-F-RNA molecules in the presence and absence of tLNAs. The 2′-F-RNA molecules have sequences designed to be complementary to several tLNAs in contiguous fashion. In the absence of interfering tLNAs, a homogeneous population of 2′-F-RNA migrates in a non-denaturing gel based on its mobility of its secondary structure. However, in the presence of tLNAs, if hybridization takes place with complementary strands of RNA, the secondary structure will be disrupted, resulting in reduced mobility of RNA as seen as a gel-shift.

The 2′-hydroxyl group of natural RNA is chemically labile, making it susceptible to internucleotide cleavage. The rate of degradation increases in the presence of divalent metal ions such as magnesium and to a greater extent with lead. This occurs at a fairly rapid rate as compared to DNA, which is devoid of the 2′-hydroxyl group. The 2′-F-RNA described herein is also devoid of the 2′-OH and as a consequence is not susceptible to degradation as is 2′-OH-RNA. However, the possibility exists that the combination of metal ions and functional groups used to assemble the aptabodies as described herein may contribute to unforeseen degradation of 2′-F-RNA. To verify the existence of intact 2′-F-RNA molecules, a denaturing gel is run to analyze the presence or absence of any degradation products before proceeding with any of the subsequent experiments. If degradation is noticed, then any metal ions and/or tLNAs responsible for the degradation will be eliminated from the aptabody assembly buffer by the process of elimination.

### Selection

The literature is replete with examples of in vitro selection of aptamers, however, selection of aptamers in whole-animals as in the present invention has never been undertaken. The conventional approach is to find an aptamer against a target of interest using in vitro techniques. Once aptamer(s) are successfully enriched through in vitro selection, any pharmacologically promising aptamer(s) are then tested in animals. However, as discussed above, aptamer drug candidates that are successful in vitro may fail in animal studies⁴⁰. In this example, we use selection of aptabodies directly in a mouse model.

Whole-animal selection for aptabodies that bind to metastatic tumors can be carried out using repeated rounds of negative and positive selection. Before positive selection, a negative selection (or pre-selection) is performed to eliminate effector-independent molecules that are active (i.e., background noise). This is accomplished by pre-exposing the aptabody library (˜10¹⁴ molecules) to a normal, healthy mouse. Long pre-exposure times for negative selection will reduce or eliminate molecular constructs that are non-specifically bound to normal tissue. Any molecules that are bound during this period will be bound sufficiently long enough to their nonspecific substrate to effectively separate those molecules from the rest of the library pool. Following negative selection, positive selection in the animal tumor model will take place, followed by tumor isolation and recovery of any aptabody molecules associated with the tumor.

### Administration of Aptabody Library to Animal Model

A healthy mouse can be anesthetized using standard methods, and surgically prepared for whole library administration and blood collection of library molecules. The entire library solution (no more than 500 μl in PBS buffer, pH 7.4) is intravenously infused in a single dose. Twenty to sixty minutes after dose administration, a blood sample of 500 μl is drawn (orbital sinus puncture) and collected in a siliconized tube to prevent blood coagulation.

Negative selection can be carried out prior to positive selection, to eliminate molecules from the pool that are bound nonspecifically to the vasculature, organs, or tissues of a normal healthy mouse. The length of exposure of the library pool to a normal healthy mouse may be varied to adjust the level of stringency for negative selection. As selection progresses, the duration of the exposure period may be progressively increased from twenty minutes for each round of selection to increase stringency and, therefore, increasingly resulting in the discarding of molecules that exhibit slow acting absorption kinetics. However, liver metabolism of aptabody molecules may significantly diminish the population at longer durations, so increasing the duration of exposure within the bloodstream may be counterproductive. Moreover, because the size of aptabodies is at least 33 kDa, renal accumulation may occur if aptabodies are not metabolized by the liver first. To circumvent the counterproductive effects of liver metabolism and renal accumulation, more than one negative selection may be carried out before positive selection. Briefly administrating and withdrawing blood samples to several mice consecutively will alleviate the molecular burden on any one particular mouse. Multiple negative selections are also beneficial to eliminate molecules from the population that are able to survive selection without themselves being specific to the target. To counter this, more than one negative selection step prior to positive selection may be necessary to rid the population of these molecules.

Immediately following the negative selection step, the blood sample is intravenously administered to another mouse with the same blood type and which has been prepared for positive selection (e.g., a mouse tumor model; see above for examples). Following a twenty minute period, the animal is sacrificed and prepared for the recovery of bound aptabody molecules.

As selection progresses, it may be desirable to decrease the length of time the mouse tumor model is exposed to the library and/or decrease the concentration of the library to avoid complete regression of tumors.

### Recovery, Amplification, and Reconstitution of Aptabody Molecules

At the end of every round of selection, mice are snap-frozen in liquid nitrogen while in a state of deep anesthesia. Following sacrifice of the animal and surgical removal of the tumor, any bound aptabodies are purified and amplified for the next round of selection. Careful excision and handling of tissue is carried out so as to avoid any potential loss of aptabodies. This is important because molecular species may exist that are weakly bound to the tissue substrate⁴¹. For example, there may be active aptabody molecules that exhibit transitional binding through weak ionic and van der Waals forces that can be easily broken. Alternatively, laser capture microdissection (LCM) may be used to isolate ultrafine regions of target tissue or subcellular regions (Arcturus, Mountain View, Calif.). The excised tumor is placed in a microcentrifuge tube and finely minced with scalpel blades or homogenized with a homogenizer. The tissue sample is disrupted in a lysis solution (e.g., NaOH/SDS) and RNA is extracted using an acid phenol:chloroform treatment followed by a glass fiber filter procedure. This procedure is commercially available in a kit form, the mirVana™ miRNA Isolation Kit (Ambion, Inc.). Unlike other commonly used RNA isolation procedures, the kit is optimized to isolate small RNAs from tissues. The extracted RNA material is resuspended in 10 μl of water.

The total 10 μl sample of RNA is reverse-transcribed using MMLV RT (Epicentre, Madison, Wis., U.S.A.) according to the manufacturer's directions with primer APTA-R (5′-GGGCAACCTACGGCTTTCACCGTTTCG-3′). This reaction generates cDNAs for subsequent PCR amplification of surviving nucleic acid sequences coding for 2′-F-RNA scaffolds. The reaction conditions are optimized such that the APTA-RT primer readily anneals and extends the cDNA at an elevated temperature such that it out competes the inhibitory effects of any tLNAs hybridized on the RNA scaffold. Amplification of cDNA is carried out using qPCR (quantitative PCR) in a real-time thermocycler. In the presence of a fluorescent dye, SYBR Green, the amplification reaction can be followed in real-time without the use of post gel analysis of amplification products. qPCR also reveals quantitative information regarding the amount of starting material which is theoretically sensitive down to one copy of cDNA. This technique is ideally suited for in vivo selection since molecules in a tissue sample may be rare and present in minute amounts. Maintaining constant reaction conditions from one round of selection to the next, a relative comparison is made of the amount of aptabodies specifically targeting tissue, which is an indicator whether the experiment is proceeding successfully over the course of selection. For every round of selection, relative amounts of aptabodies present in tissue samples are compared to a standard curve generated by a serial dilution of a known volume (e.g., 2 μl) of library taken just prior to the bolus administration in an animal model. The threshold cycle number (C_{T}), or the minimum number of PCR cycles where enough amplified product accumulates to yield a detectable fluorescent signal, of test samples are compared to standards to quantitatively assess the fraction of the bolus administered library that has found its way to the target phenotype (e.g., tissue). The single-step RT-PCR conditions are as follows: 1 μl of test sample is added to a 19 μl reaction mix (in a glass capillary tube) consisting of the final concentrations of 3.25 mM Mn(OAc)₂; 0.3 μM primers APTA-T7 and APTA-RT; 1× of Lightcycler RNA Master SYBR Green I mix from kit #03064760001 (Roche, Mannheim, Germany). The master mix contains a propriety blend of hot-start Taq polymerase with thermostable polymerase and reverse transcriptase activity, magnesium buffer, dNTPs, SYBR Green I fluorescent dye. The thermocycler (Lightcycler, Roche) is programmed for reverse transcription at 61° C. for 20 minutes; denaturation at 95° C. for 30 seconds; followed by PCR amplification. All temperature transition rates are 20° C./second unless otherwise specified. Generally, 10-15 cycles of PCR are needed under the following cycling parameters: 95° C. for 1 second, 55° C. for 5 seconds, and 72° C. for 5 seconds. Melting curve analysis parameters are as follows: 95° C., 65° C., 95° C. (slope of 0.1° C./second); final cooling is at 40° C. for 30 seconds. Proper negative and positive controls are used in every RT-PCR reaction run, i.e., water sample and library standards, respectively. Following RT-PCR, ¼ of the sample may be run on an analytical agarose gel to verify amplified product integrity; another ¼ sample is transcribed as described above, combined with tLNAs to reconstitute an enriched aptabody pool, and subjected to another round of selection.

### Monitoring the Progress of Selection

Iterative cycles of selection and amplification are repeated until a significant proportion of the aptabody population consists of molecules that preferentially absorb in the tumor-induced mouse versus nonspecifically in a normal, healthy mouse without tumors. The fraction of the aptabody library absorbed in presence of a tumor (+ effector) relative to the fraction absorbed without a tumor (− effector) is determined from the mice used in the positive and negative selection, respectively. A fraction greater than one signifies that a majority of aptabody molecules within the library preferentially absorb in the presence of effector versus in the absence of effector. At the point at which the fraction absorbed in the tumor mouse (signal) is several times greater than that in the normal, healthy mouse (background), selection is stopped and the library population is cloned, sequenced, and characterized for anti-tumor activity.

Since the relative fraction absorbed in the presence and absence of effector is used to gauge the relative level of specificity, the measurement of absolute absorption in mice is not needed. Instead, a simpler method of relative absorption is determined. If X1 and Y1 represent the amount of library pool just prior to administration in the negative and positive animal models, respectively, and X2 and Y2 represent the amount of library pool from the blood withdrawn twenty minutes later from the negative and positive animal models, respectively, then the relative fraction absorbed in the presence versus absence of tumor is:

$$\frac{( Y 1 - Y 2 ) / Y 1}{( X 1 - X 2 ) / X 2}$$

The twenty minute time interval and sample volumes are kept constant from one mouse to the next to establish with assurance the signal-to-noise ratio for each round of selection. Note, this is a relative measure and does not take into account liver metabolism and renal accumulation of molecules as discussed previously. However, the rates for these parameters are assumed to be constant across each mouse, thereby canceling each other out.

As selection progresses, the signal-to-noise ratio is expected to increase, which signifies that the population is being progressively enriched with aptabody molecules that preferentially absorb within the tumor mouse model. However, if three consecutive rounds of selection show no change in the signal-to-noise ratio, then the stringency of negative selection can be increased. No more than fifteen rounds of selection are typically performed in this experiment, unless there is significant improvement in the signal-to-noise ratio from one round to the next.

### Post-Selection Analysis

Molecular species in the aptabody library may exist that display high binding affinity to the target tumors. However, these species may be pharmacologically inactive with respect to the tumors when bound. The negative and positive selection in this investigation is designed to select for aptabodies that prevent or allow absorption of aptabodies in their respective animal models. It does not address or account for the selection of anti-tumor activity. Therefore, we must sort through the population in the final round of selection to screen for pharmacologically active clones.

The PCR product in the final round of selection is cloned (TOPO TA cloning Kit, Invitrogen Corporation, Carlsbad, Calif., U.S.A.) and sequenced (ThermoSequenase Cycle Sequencing Kit, USB Corporation, Cleveland, Ohio, U.S.A.) to generate an artificial phylogeny. Fifteen clones are characterized. Each unique clone sequence is reconstituted as an aptabody by 2′-F-RNA transcription of the TOPO TA plasmid template followed by hybridization with tLNAs as described previously. The aptabody preparation is equally divided into five aliquots for a time-course analysis of anti-tumor activity in mouse tumor models. Since the mechanism of therapeutic action, if any, is unknown, the morphological progression of tumor regression is monitored.

Surgical procedures of mice and the inoculation of mice for tumor growth is as described above, except that the inoculant is equally divided into five separate aliquots, each of which is used to inoculate mice to generate five mouse tumor models. Each 1/5 sample of an aptabody is simultaneously administered to each of the mouse tumor models. At 0, 1, 5, 15, and 30 hours, a mouse is immediately sacrificed and pathological tissue removed for histopathological and morphometric analysis. For each mouse, a 3 μm tissue section is H&E stained, digitally photographed, and the image analyzed for various morphological features. The fraction of histomarker regression is determined for each clone at each time-point (histomarker regression=(# of abnormal cells)/(# of abnormal cells at time zero) at constant microscopic field of view).

The five most promising clones are further characterized for their relative absorption as described above. Since the population in the final round of selection has a high signal-to-noise ratio, the five best performing clones are expected to exhibit an equivalent level of absorbance characteristics.

### Phase II

A successful selection experiment may generate one or more solutions, i.e., the identification of particular RNA sequences exhibiting a desired aptabody activity. However, there may exist RNA sequences related to the molecules identified in the screen that satisfy the selection criteria, but were not represented in the initial RNA population, and therefore did not have a chance to be enriched through selection. Moreover, these unrepresented sequences may be aptabodies with attractive pharmacokinetics. For instance, the optimal aptabody solution is a species that displays a high level, specific anti-tumor activity.

To search for these other possible solutions, another selection experiment (or reselection) can be performed with an RNA library constructed from the selected parent (or wildtype) sequence that has been moderately mutated to explore other related sequences. For reselection, the wildtype sequence can be mutagenized in the non-primer region at a rate of, for example, 19% per position. A population of these constructs can be subjected to several more rounds of selection to enrich for active variants from the originally selected type sequence. The population can subsequently be cloned and sequenced, and individual clones analyzed for activity.

Additional optimization can involve investigation of absorption, distribution, metabolism, excretion, and toxicity (ADMET) issues for the most promising aptabody drug candidates, and evaluation of the biodistribution patterns of these molecules in several major organs using such techniques as whole-body autoradiography. Further studies can relate to determining the mechanisms of action of selected aptabodies. For example, the anti-tumor activity of a selected molecule may be a direct consequence of the activity of an aptabody on an affected tissue or may occur through indirect mechanisms. For instance, an aptabody drug may recruit the immune system to specifically attack a diseased tissue. Another possible mode of action is the stimulation of endogenous genetic or epigenetic factors that may contribute to the dissolution of affected tissue.

### Example II

### Molecular Switch Technology

As is noted above, the nucleic acid molecule complexes of the invention include functional groups, which, in combination with the nucleic acid components of the complexes, form molecules with desirable therapeutic and/or diagnostic properties. In addition to molecules that may not have any particular function or activity on their own, the functional groups used in the invention can also be molecules that are known drugs, which can be included in the nucleic acid molecule complexes of the invention to result in some type of improvement in their activity (e.g., increased therapeutic effect, decreased toxicity, increased bioavailability, and/or localization).

Another possible type of improvement to a drug that can be achieved using the approaches of the invention is the shielding of its activity in locations where its effect is not needed or indeed is undesirable, and exposure of its activity at a target site. In this context, the nucleic acid, aptamer component of the nucleic acid molecule complexes of the invention can be said to act as a “molecular switch,” unmasking the activity of the drug in the presence of its target, and sequestering the activity when present systemically. Drug molecules used as functional groups in this example can be drugs that were previously known to be effective against a pathological condition, but fall short of an idealized drug compound because of, e.g., non-specificity, cross-reactivity, or toxicity. The drugs can also be former drug candidates that were previously dismissed as failed candidates. Including such drugs in a nucleic acid molecule complex, as can be selected in the present invention, can result in the drug as having a useful activity.

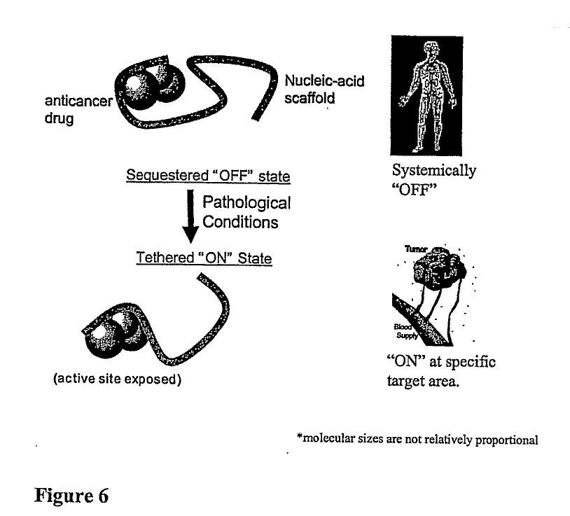

In one example of the molecular switch technology of the invention, an anticancer drug, present in the context of a nucleic acid molecule complex of the invention, is selected so that it is in an active state at the site of a tumor, but is inactive systemically (FIG. 6). In this example, the drug consists of two components: (i) a known anticancer drug coupled to and inactivated by (ii) an aptamer in its natural state. In the pathological environment, the aptamer component is released from the drug active site, allowing activity on the tumor substrate. A return to the normal, systemic environment collapses the aptamer back onto the active site, thereby preventing activity. The highly controlled on/off specificity and activity of the drug reduces or eliminates systemic toxicity, which is commonly associated with anticancer agents and results in severe limitations to their use.

Anticancer agents that can be modified so as to be under the control of a molecular switch, according to the invention, include, for example, doxorubicin. In other examples, the anticancer agent is a chemotherapeutic agent such as an alkylating agent (e.g., busulfan, caroplatin, carmustine, chlorambucil, cisplatin, cyclophosphamide (i.e., cytoxan), dacarbazine, ifosfamide, lomustine, mecholarethamine, melphalan, procarbazine, streptozocin, or thiotepa); an antineoplastic antibiotic (e.g., bleomycin, dactinomycin, daunorubicin, idarubicin, mitomycin (e.g., mitomycin C), mitoxantrone, pentostatin, or plicamycin); an antimetabolite (e.g., an thymidylate synthetase inhibitor (e.g., fluorodeoxyuridine), cladribine, cytarabine, floxuridine, fludarabine, fluorouracil (e.g., 5-fluorouracil (5FU)), gemcitabine, hydroxyurea, mercaptopurine, methotrexate, or thioguanine); or a natural source derivative (e.g., docetaxel, etoposide, irinotecan, paclitaxel, teniposide, topotecan, vinblastine, vincristine, vinorelbine, taxol, prednisone, or tamoxifen). Any of these or other agents can be included in nucleic acid molecule complexes, according to the invention, and selected for desired activities. The modified anticancer agents can be selected by, for example, monitoring an animal model of cancer for decreased systemic effects, while no change (or even improvement in) anticancer effects.

In another example of the molecular switch technology of the invention, the activity of a drug (e.g., an enzyme) is modulated by an aptamer that is sensitive to particular physiological conditions. One example of such an enzyme is tissue plasminogen activator (tPA), which is used in the treatment of stroke. Stroke is the leading cause of disability and the third leading cause of death in the United States⁴², and occurs when a blood vessel in or around the brain becomes plugged (thrombotic) or ruptures (hemorrhagic). The blocked vessel initiates an ischemic response and triggers several pathways that ultimately lead to cell death of surrounding brain tissue. In almost all cases, if the stroke is not treated, the patient becomes unconscious and succumbs. The only pharmaceutical approach currently approved by the United States FDA involves the use of a class of thrombolytics, such as tissue plasminogen activator (tPA), which is a fibrinolytic serine protease that has been found in many tissues and body fluids. tPA works by converting plasminogen to plasmin, which then dissolves fibrin, a major component of blood clots.

The most serious drawback to tPA therapy is systemic fibrinogen degradation. Because fibrinogen is found throughout the body in capillary membranes and scar tissue, current tPA drugs are limited in use because they dissolve all fibrinogen structures leading to a potentially high risk of systemic hemorrhagic bleeding. Moreover, after a stroke episode, thrombolytics are only effective within 3 hours, and the dosage must be closely monitored. For this reason, only about 1 in 20 patients receives tPA, because most stroke victims get to a hospital more than 3 hours after they have been stricken. By then, the risk of hemorrhage from a dose of tPA outweighs the potential benefits of the drug.

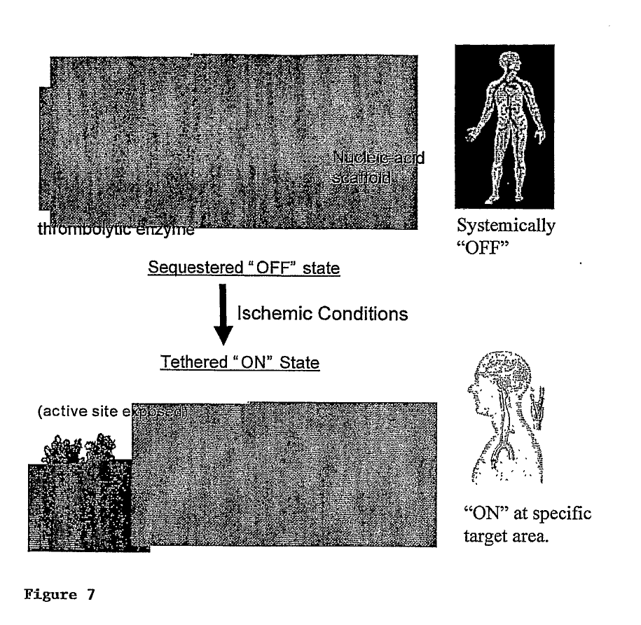

Thus, tPA is an example of a drug that can be modified, according to the invention, to improve its therapeutic properties. In particular, the drug can be improved so that it is active under conditions in which a clot may occur (e.g., ischemic conditions) and be inactive systemically (FIG. 7). In this example, the drug consists of two components: (i) a thrombolytic enzyme (e.g., tissue plasminogen activator (tPA)) coupled to and inactivated by (ii) an aptabody in its natural state. In an ischemic environment, the aptabody component is released from the thrombolytic enzyme active site, allowing catalytic activity on the clot substrate. A return to unimpeded blood flow collapses the aptabody back onto the active site, preventing enzymatic activity. The highly controlled on/off specificity and activity of the drug reduces or eliminates systemic hemorrhagic bleeding, which is commonly associated with current, non-specific drugs.

Drugs including such molecular switches can be selected based on detection of decreased systemic effects, coupled with improved effects in target areas. The molecular switch aptabodies of the invention are feasible, because of physiological factors that are known to be associated with a target area. In the case of a thrombolytic-aptabody switch, as described above, although the triggering factor(s) for activation of tPA at the clot area may not be known without analyzing the results of a successful selection and deducing the possible mechanism of action, predictions can be made based on previous evidence suggesting the presence of one or more physiological factors that are associated with formed clots and the surrounding area. One or more of these factors may be used as an effector(s) for triggering the release of tPA from its inactive to active state. One example of a possible effector is fibrin. The thrombolytic desmoteplase derived from vampire bat saliva is highly specific to fibrin, with its activity being 102,000 times higher in the presence of fibrin than in its absence, whereas tPA is specifically enhanced only 72-fold by fibrin. Engineering an aptabody that augments tPA by exposing the active site of tPA and blocking it in the presence and absence of fibrin, respectively, will enhance tPA-aptabody to the status of desmoteplase.

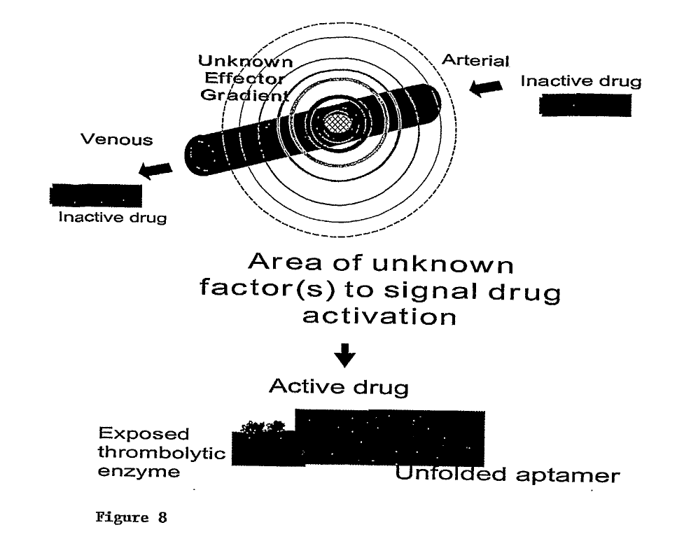

The folding of nucleic acid molecules is sensitive to a variety of environmental factors, including salt, pH, divalent ions, and temperature. As a result, nucleic acid-based aptabodies that bind to their targets can be selected to be responsive to any one or a combination of such factors. For instance, another plausible effector is the local pH surrounding a thrombus clot. Ischemia surrounding a clot causes a pH drop in the tissue with deficient blood supply⁴³. The serum pH in the vicinity of a clot is lowered, due to ischemia-induced accumulation of lactic and pyruvic acids in the surrounding local area. If an allosteric tPA is selected such that the activity increases as lactic or pyruvic acids increase or alternatively as the general pH of the area decreases, then the selected enzyme will have its greatest effect at the site of the clot, and minimal or no activity elsewhere. Other potential effectors surrounding ischemic sites include ATP and glucose, which vary in concentration depending on blood flow⁴⁴. There are likely a host of unknown physiological factors that can allow for a clot-specific allosteric enzyme switch (FIG. 8).

Molecular species in the aptabody library may exist that display ideal on-off dynamics. However, these species may be tPA inactive when bound to the target clot. The negative and positive selection steps of the present invention can be used to select for aptabodies that prevent or allow absorption of tPA in their respective animal models. Molecules that survive both negative and positive selection may exist where the active site of tPA is blocked yet the aptabody binds specifically to the thrombus clot. Then, in later steps complexes can be selected for thrombolytic activity.

A successful selection experiment may generate one or more active clone solutions, i.e., particular nucleic acid molecule complexes having a desired activity. However, there may be a potential for other complexes, having RNA sequences related to these parent solutions that satisfy the selection criteria, but were not represented in the initial RNA population, and therefore did not have a chance to be enriched through selection. Moreover, these unrepresented sequences may be aptabodies with attractive pharmacokinetics or [drug]-aptabody switches with attractive on/off kinetic performance. For instance, the optimal aptabody solution is a species that displays a high specific anti-tumor activity or as in the case for [drug]-aptabody is a species that displays all-or-none activity. In the presence of tumor, the switch turns completely on and is completely off otherwise. The closer we are to achieving this ideal solution, the better the therapeutic profile of the drug. Such molecules can be identified using additional steps of mutagenesis and selection, as described above.
¹Wagner, R. W. et al., Potent and selective inhibition of gene expression by an antisense heptanucleotide. Nature Biotechnology, 1996, 14 (Jul.): 840-844.
²Mullis, K B, Target Amplification For DNA Analysis By The Polymerase Chain Reaction. Ann. Biol. Clin-Paris, 1990, 48 (8): 579-582.
³Schena M, et al. Quantitative Monitoring of Gene-Expression Patterns with a Complementary-DNA Microarray. Science, 1995, Oct. 20. 270 (5235): 467-470.
⁴Shoemaker D D, et al., Experimental Annotation of the Human Genome using Microarray Technology. Nature, 2001, Feb. 15, 409 (6822): 922-+.
⁵Bischofberger, N. and R. G. Shea, Oligonucleotide-Based Therapeutics. Nucleic Acid Targeted Drug Design, C. L. Propst and T. J. Perum, Editors. 1992, Marcel Dekker, Inc., New York: 579-613.
⁶Bartel, D. P. and J. W. Szostak, Study of RNA-protein recognition by in vitro selection. RNA-Protein Interactions, K. Nagai and I. W. Mattaj, Editors, 1994, IRL Press, New York: 248-263.
⁷Sun, F., D. Galas, and M. S. Waterman, A Mathematical Analysis of in Vitro Molecular Selection-Amplification. Journal of Molecular Biology, 1996. 258: 650-660.
⁸Gold, L., Conformational properties of oligonucleotides. Nucleic Acids Symposium Series, 1995 (No. 33): 20-22.
⁹Wlotzka, B., et al., In vivo properties of an anti-GnRH Spiegelmer: an example of an oligonucleotide-based therapeutic substance class. Proc. Natl. Acad. Sci. USA, 2002, 99:8898-8902.
¹⁰Drolet, D W., et al., Pharmacokinetics and safety of an antivascular endothelial growth factor aptamer (NX1838) following injection into the vitreous humor of rhesus monkeys, Pharm. Res., 2003, 17: 1503-1510.
¹¹D. Nieuwlandt, et al. In Vitro Selection of RNA Ligands to Substance P. Biochemistry, 1995, 34: 5651-5659.
¹²Q. Wu, et al. Localization of the single-stranded DNA Binding Site in the Thrombin Anion-binding Exosite. The Journal of Biological Chemistry, 1992, 267 (No. 34, December 5): 24408-24412.
¹³D. W. Drolet, et al. An enzyme-linked oligonucleotide assay. Nature Biotechnology, 1996. 14 (Aug.): 1021-1026.
¹⁴Beuckelaer, A D and J. P. FArste, Selection of high affinity RNA ligands to a synthetic peptide of human rhinovirus 14 coat protein. Nucleic Acids Symposium, 1995 (No. 33): 23.
¹⁵Nielson, P E, Sequence-Selective DNA Recognition by Synthetic Ligands. Bioconjugate Chemistry, 1991, 2 (1) January/February: 1-12.
¹⁶Li W X, et al., A novel nucleotide-based thrombin inhibitor inhibits clot-bound thrombin and reduces arterial platelet thrombus fommation., Blood, 1994, 83: 677-682.
¹⁷Rusconi C P, et al., RNA aptainers as reversible antagonists of coagulation factor Ixa. Nature 2002, 419: 90-94.
¹⁸Gold, L., Oligonucleotides as Research, Diagnostic, and Therapeutic Agents. The Journal of Biological Chemistry, 1995. 270 (No. 23, June 9): 13581-13584.
¹⁹Trotta, P. P., B. A. Beutel, and M. I. Sherman, Oligonucleotide Diversity and Drug Discovery. Medicinal Research Reviews, 1995. 15 (No. 4): 277-298.
²⁰McGown, L. B. et al., The Nucleic Acid Ligand: A New Tool for Molecular Recognition. Analytical Chemistry, 1995 (November 1): 663A-668A.
²¹Gold, L., et al., Diversity of Oligonucleotide Functions. Annual Review of Biochemistry, 1995, 64: 763-797.
²²Nielsen, ed., Peptide Nucleic Acids: Protocols and Applications (First Edition), Horizon Scientific Press, 1999.
²³Hsaio et al., Science 274:99-102, 1996.
²⁴Lewis et al., Nat. Gen. 25:402-406, 2000.
²⁵Masliah et al., Science 287 (5456): 1265-1269, 2000.
²⁶Giasson et al., Neuron 34:521-533, 2002.
²⁷Sommer et al., Exp. Geront. 35:1389-1403, 2000.
²⁸Lewis et al., Nat. Genet. 25 (4):402-405, 2000.
²⁹Takahashi et al., Res. Commun. Chem. Pathol. Pharmacol. 66 (1):167-170, 1989.
³⁰Petroske et al., Neuroscience 106 (3):589-601, 2001.
³¹Przedborski et al., Neuroscience 67 (3):631-647, 1995.
³²Brooks et al., Brain Res. 823 (1-2):1-10, 1999.
³³Thiruchelvam et al., J. Neurosci. 20 (24):9207-9214, 2000.
³⁴Mihm et al., J. Neurosci. 21 (11):RC149, 2001.
³⁵Leppanen et al., American Heart Association 20 (11):e89-e95, 2000.
³⁶Remington's Pharmaceutical Sciences (18^{th} edition), ed. A. Gennaro, 1990, Mack Publishing Company, Easton, Pa.
³⁷Pagratis, N. C., et al., Potent 2′-amino-, and 2′-fluoro-2′-deoxyribonucleotide RNA inhibitors of keratinocyte growth factor, Nature biotechnology, 1997, 15:68-73.
³⁸Scmidt, Kathrin s., et al., Application of locked nucleic acids to improve aptamer in vivo stability and targeting function, Nucleic Acids Research, 2004, 32 (19): 5757-5765.
³⁹Knudsen, Scott M., et al., In Vitro Selection Using Modified or Unnatural Nucleotides, Current Protocols in Nucleic Acid Chemistry, 2001, unit 9.6.1-9.6.21.
⁴⁰James, William, Encyclopedia of Analytical Chemistry, John Wiley & Sons, 2000, 4848-4871.
⁴¹Stumpf, Walter E., Drug localization and targeting with receptor microscopic autoradiography, Journal of Pharmacological and Toxicological Methods, 2005, 51: 25-40.
⁴²Alberts, Mark J., Diagnosis and Treatment of Ischemic Stroke, The American Journal of Medicine, 1999, 106: 211-221.
⁴³Suchkova, V N, et al. Effect of 40-kHz ultrasound on acute thrombotic ischemia in a rabbit femoral artery thrombosis model. Circulation, 2000, 101: 2296-2301
⁴⁴ Busch, Elmar, et al., Reperfusion After Thrombolytic Therapy of Embolic Stroke in the Rat: Magnetic Resonance and Biochemical Imaging, Journal of Cerebral Blood Flow and Metabolism, 1998, 18 (4): 407-418.

1. A method for identifying one or more candidate therapeutic or diagnostic molecules, the method comprising the steps of:
(i) introducing a population of nucleic acid molecule complexes into a test subject exhibiting a phenotype of a disease or condition, wherein the nucleic acid molecule complexes each comprise
(a) a first nucleic acid molecule that comprises a segment of random sequences, and
(b) a second nucleic acid molecule that comprises (i) a nucleic acid molecule segment that hybridizes to the first nucleic acid molecule, and (ii) a functional group conjugated to the nucleic acid molecule segment, wherein the nucleic acid molecule segment comprises one or more nucleotides that establish a fixed spatial orientation of the functional group with respect to the nucleic acid segment; and
(ii) isolating from the test subject those nucleic acid molecule complexes that localize to a tissue exhibiting a phenotype characteristic of the disease or condition,
thereby identifying nucleic acid molecule complexes that are candidate therapeutic or diagnostic molecules for the disease or condition.

2. The method of claim 1, wherein the disease phenotype is characterized by naturally occurring or artificially induced phenotypic markers that reveal anatomical, physiological, cellular, or receptor structures.

3. The method of claim 2, wherein determining whether a nucleic acid molecule complex has localized to a tissue exhibiting a phenotype characteristic of the disease or condition comprises detection of a marker by use of imaging reagents.

4. The method of claim 3, wherein the marker is selected from the group consisting of biological, histochemical, immunological, chemiluminescent, physiochemical, radioisotope, photochemical, and electromagnetic markers.

5. The method of claim 1, wherein the method further comprises the step of contacting a population of first nucleic acid molecules with a population of second nucleic acid molecules under conditions that promote hybridization, to generate the nucleic acid molecule complexes that are administered to the test subject.

6. The method of claim 1, further comprising introducing the nucleic acid molecule complexes into a healthy subject, discarding any of the complexes that remain bound or absorbed in the healthy subject, and introducing any remaining complexes into the test subject.

7. The method of claim 1 or claim 6, further comprising, after isolation of the complexes, (i) incubating the complexes under conditions that disrupt the hybridization between the first and second nucleic acid molecules, and (ii) amplifying the isolated first nucleic acid molecules to generate an amplified, selected pool of first nucleic acid molecules.

8. The method of claim 7, further comprising contacting the amplified, selected pool of first nucleic acid molecules with a population of second nucleic acid molecules to generate nucleic acid molecule complexes for further in vivo selection.

9. The method of claim 1, further comprising determining the ratio of absorption of nucleic acid molecule complexes in the test subject as compared to a healthy subject, wherein a ratio of greater than one indicates the identification of candidate therapeutic or diagnostic molecules for the disease or condition, which may be subject to further selection.

10. The method of claim 1, further comprising subjecting candidate therapeutic or diagnostic molecules identified according to the method to mutagenesis, followed by further in vivo selection, to identify additional candidate therapeutic or diagnostic molecules.

11. The method of claim 1, further comprising testing the isolated nucleic acid molecule complexes for efficacy as therapeutic or diagnostic agents.

12. The method of claim 1, further comprising identifying a single, isolated complex as a therapeutic or diagnostic molecule.

13. The method of claim 1, wherein the first nucleic acid molecules each comprise constant segments that flank the random sequences and facilitate amplification.

14. The method of claim 1, wherein the first nucleic acid molecules each comprise nucleotides which are stable from degradation or elimination in vivo, prior to achieving a therapeutic or diagnostically detectable effect.

15. The method of claim 14, wherein the first nucleic acid molecules comprise a
nucleic acid analog or derivative.

16. The method of claim 15, wherein the nucleotide analog is selected from the group consisting of phosphorothioate, boranophosphate, methyl-phosphonate, and 2′-O-methyl analogs, and analogs thereof.

17. The method of claim 16, wherein the analog is 2′-deoxy-2′-fluoro-RNA (2′-F-RNA).

18. The method of claim 1, wherein the second nucleic acid molecules each comprise a locked nucleic acid molecule (LNA), a peptide nucleic acid molecule (PNA), a morpholino oligonucleotide, or another type of DNA or RNA molecule.

19. The method of claim 1, wherein the functional groups of the second nucleic acid molecules are independently selected from the group consisting of amino acids, fatty acids, polyethylene glycols, carbohydrates, toxins, small organic molecules, drug compounds, vitamins, cofactors, inorganic compounds, metals, elements of the periodic table and isotope forms thereof, and aggregate or higher-order forms thereof.

20. The method of claim 19, wherein the aggregate or higher-order forms comprise peptides, proteins, lipids, complex sugar moieties, or chelated metals.

21. The method of claim 1, wherein the functional groups of the second nucleic acid molecules are independently selected from the group consisting of cytokines, immune response factors, antibodies, complement components, hormones, neurotransmitters, naturally occurring chemicals, and biological macromolecules.

22. The method of claim 1, wherein the nucleic acid molecule complexes are formed in the presence of one or more metal ions, or one or more organic or inorganic co-factors.

23. The method of claim 22, wherein the one or more metal ions are selected from the group consisting of MgCl₂, FeCl₂, CuCl₂, KCl, LiCl, NaCl, RbCl, CaCl₂, SrCl₂, CdCl₂, CoCl₂, MnCl₂, NiCl₂, ZnCl₂, and other elemental salts.

24. The method of claim 1, wherein the test subject is an animal model of the disease or condition, or a patient having the disease or condition.

25. The method of claim 24, wherein the test subject is an animal model of the disease or condition.

26. The method of claim 24, wherein the test subject is a patient having the disease or condition.

27. The method of claim 26, wherein the patient is a human patient.

28. The method of claim 24, wherein the disease or condition is pathological, physiological, psychological, behavioral, or cognitive in origin.

29. The method of claim 24, wherein the disease or condition is selected from the group consisting of cell, tissue, or organ injury, cytopathology, inflammation, repair, regeneration, and fibrosis, immunopathology, neoplasia, developmental disorders, hemodynamic disorders, nutritional pathology, and diseases or conditions affecting the blood vessels, heart, respiratory system, gastrointestinal tract, liver and biliary system, pancreas, kidney, male or female reproductive systems, breast, blood and lymphoid organs, endocrine system, embryonic system, skin, head and neck, bones and joints, skeletal muscle, nervous system and brain, eye and sensory systems such as vision, taste, smell, hearing, and touch.

30. The method of claim 24, wherein the disease or condition is genetically based, or due to a viral, bacterial, microbial, protozoal, parasitic, or fungal infection.

31. The method of claim 24, wherein the disease or condition is due to diet, poison, toxins, heavy metals, or exposure to environmental hazards.

32. The method of claim 24, wherein the disease or condition is selected from the group consisting of cancer, such as Non-Hodgkin's lymphoma, leukemia, melanoma, lung cancer, pancreatic cancer, stomach cancer, throat cancer, and glioblastoma, Gaucher disease, Hepatitis B and C, respiratory syncytial virus infection, diabetes, obesity, neurological diseases, such as Alzheimer's disease and Parkinson's disease, cardiovascular diseases, such as coronary artery disease, myocardial ischemia, stroke, pulmonary embolism, restenosis, and hypertension, tuberculosis, multiple sclerosis, anaemia, respiratory diseases, arthritis, Crohn's disease, cystic fibrosis, neutropaenia, malaria, general or localized pain, depression, anxiety, psychiatric disorders such as schizophrenia, epilepsy, narcolepsy, sleep disorders, language disorders and aphasias, learning and memory disorders, degenerative disorders, drug addiction, asthma, allergies, gout, hypertension, hypercholesterolemia, dyslipidemia, peptic ulcers, gastroesophageal reflux disease, inflammatory bowel disease, urinary tract infections, and complications of these diseases or conditions.

33. The method of claim 12, wherein the therapeutic molecule is selected from the group consisting of enzymes, molecular switches, receptor agonists or antagonists, ion channel modulators, anesthetics, analgesics, hypnotics, sedatives, hallucinogenics, cognitive enhancing agents, anti-inflammatory agents, antipyretics, diuretics, antihypertensives, antiarrhythmics, prokinetics, antiemetics, antimicrobials, antifungals, antivirals, antiretrovirals, antineoplastics, immunomodulators, immunosuppresives, immunostimulants, hematopoietic agents, anticoagulants, thrombolytics, antiplatelet agents, antithyroid agents, hormone agonists or antagonists, hormonal-mimetics, dermatological agents, opthalmological agents, toxicological agents, antivenoms, and antidotes.

34. A nucleic acid molecule complex comprising: (i) a first nucleic acid molecule, and (ii) a second nucleic acid molecule hybridized thereto, wherein the second nucleic acid molecule comprises one or more nucleotides that establish a fixed spatial orientation of the functional group with respect to the nucleic acid segment of the second nucleic acid molecule and the second nucleic acid molecule further comprises a functional group conjugated to the second nucleic acid molecule.

35. The nucleic acid molecule complex of claim 34, wherein the first nucleic acid molecule comprises nucleotides which are stable from degradation in vivo, prior to achieving a therapeutic or diagnostically detectable effect.

36. The nucleic acid molecule complex of claim 35, wherein the first nucleic acid molecules comprise a nucleic acid analog or derivative.

37. The nucleic acid molecule complex of claim 36, wherein the nucleotide analog is selected from the group consisting of phosphorothioate, boranophosphate, methyl-phosphonate, and 2′-O-methyl analogs, and analogs thereof.

38. The nucleic acid molecule complex of claim 37, wherein the analog is 2′-deoxy-2′-fluoro-RNA (2′-F-RNA).

39. The nucleic acid molecule complex of claim 34, wherein the second nucleic acid molecules each comprise a locked nucleic acid molecule (LNA), a peptide nucleic acid molecule (PNA), a morpholino oligonucleotide, or another type of DNA or RNA molecule.

40. The nucleic acid molecule complex of claim 34, wherein the functional groups of the second nucleic acid molecules are independently selected from the group consisting of amino acids, fatty acids, polyethylene glycols, carbohydrates, toxins, small organic molecules, drug compounds, vitamins, cofactors, inorganic compounds, metals, elements of the periodic table or isotopic forms thereof, and aggregate or higher-order forms thereof.

41. The nucleic acid molecule complex of claim 40, wherein the aggregate or higher-order forms comprise peptides, proteins, lipids, complex sugar moieties, or chelated metals.

42. The nucleic acid molecule of claim 34, wherein the functional groups of the second nucleic acid molecules are independently selected from the group consisting of cytokines, immune response factors, antibodies, complement components, hormones, neurotransmitters, naturally occurring chemicals, and biological macromolecules.

43. The nucleic acid molecule complex of claim 34, wherein the nucleic acid molecule complexes are formed in the presence of one or more metal ions, or one or more organic or inorganic co-factors.

44. The nucleic acid molecule complex of claim 43, wherein the one or more metal ions are selected from the group consisting of MgCl₂, FeCl₂, CuCl₂, KCl, LiCl, NaCl, RbCl, CaCl₂, SrCl₂, CdCl₂, CoCl₂, MnCl₂, NiCl₂, ZnCl₂, and other elemental salts.

45. The nucleic acid molecule complex of claim 34, wherein the nucleic acid molecule complex is identified using the method of claim 1.

46. A method of preventing, treating, or diagnosing a disease or condition in a patient, the method comprising administering to the patient a nucleic acid molecule complex identified using the method of claim 1.

47. The method of claim 46, wherein the nucleic acid molecule complex was further identified as reducing a marker of the disease in an animal model of the disease.

48. A method of enhancing a condition in a patient, wherein the condition is selected from the group consisting of muscle strength, sexual potency, energy levels, emotional stability, and cognitive or memory capacity, the method comprising administering to the patient a nucleic acid molecule complex identified using the method of claim 1.
